(19) United States
(12) Patent Application Publication
(10) Pub. No.: US 2008/0166310 A1
(43) Pub. Date: Jul. 10, 2008

(54) Administration of 2-oxyacetamide compounds for promoting and/or inducing and/or stimulating the pigmentation of keratin materials and/or for limiting the depigmentation and/or bleaching thereof

(75) Inventors: Roger Rozot, Lagny/Marne (FR); Philippe Breton, Noisy Le Roi (FR); Michel Neuwels, Waterloo (BE); Christophe Boulle, Lagny/Marne (FR)

Correspondence Address: BUCHANAN, INGERSOLL & ROONEY PC, POST OFFICE BOX 1404, ALEXANDRIA, VA, 22313-1404, US

(73) Assignee: L'OREAL, PARIS (FR)

(21) Appl. No.: 12/000,180

(22) Filed: Dec. 10, 2007

Related U.S. Application Data
(63) Continuation of application No. PCT/EP06/05327, filed on May 12, 2006.
(60) Provisional application No. 60/689,537, filed on Jun. 13, 2005.

(30) Foreign Application Priority Data
Jun. 9, 2005 (FR) 0551554

(51) Int. Cl. A61K 8/42; A61K 8/49; A61K 31/47; A61K 31/24; A61K 31/165; A61K 31/341; A61K 31/428; A61K 31/4164; A61K 31/40; A61Q 5/00

(52) U.S. Cl. 424/70.1; 514/311; 514/535; 514/622; 514/471; 514/367; 514/399; 514/427

(57) ABSTRACT

A regime or regimen for promoting and/or inducing and/or stimulating the pigmentation of keratin materials and/or for preventing and/or limiting the depigmentation and/or bleaching and/or for preventing and/or limiting the canities thereof, more particularly of human keratin fibers such as the hair, beard hair, moustache hair, the eyelashes and the eyebrows, includes administering to an individual in need of such treatment at least one 2-oxyacetamide compound of formula (I), or a salt and/or solvate thereof:

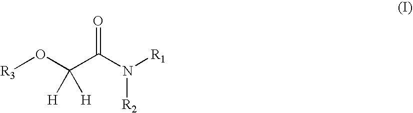

### CROSS-REFERENCE TO PRIORITY/PCT/PROVISIONAL APPLICATIONS

This application claims priority under 35 U.S.C. § 119 of FR 05/51554, filed Jun. 9, 2005, and of Provisional Application No. 60/689,537, filed Jun. 13, 2005, and is a continuation of PCT/EP 2006/005327, filed May 12, 2006 and designating the United States, published in the English language as WO 2006/131282 A1 on Dec. 14, 2006, each hereby expressly incorporated by reference in its entirety and each assigned to the assignee hereof.

### BACKGROUND OF THE INVENTION

1. Technical Field of the Invention

The present invention relates to the cosmetic administration of at least one 2-oxyacetamide compound as an agent for promoting and/or inducing and/or stimulating the pigmentation of keratin materials and/or for limiting the depigmentation and/or bleaching thereof, and more particularly as an agent for preventing and/or limiting canities of human keratin fibers.

The keratin materials to which this invention relates include human skin and nails, and human keratin fibers such as the hair, the eyebrows, the eyelashes, beard hair, moustache hair and pubic hairs. This invention also relates to the keratin materials of mammalian animals (for example dogs, horses or cats). More especially, the present invention relates to human hair, beard hair, moustache hair, eyelashes and eyebrows.

2. Description of Background and/or Related and/or Prior Art

Need exists for novel products for caring for and/or treating human keratin materials that promotes their pigmentation and/or limits their depigmentation, and more particularly for products that can prevent and/or reduce canities of human keratin fibers such as the hair, the eyelashes and/or certain bodily hairs.

The color of human hair and skin depends on various factors and especially on the seasons of the year, race, sex and age. It is mainly determined by the concentration of melanin produced by the melanocytes. These melanocytes are specialized cells that synthesize melanin via particular organelles, the melanosomes.

Melanin synthesis (or melanogenesis) is complex and schematically entails the following main steps:

Tyrosine->Dopa->Dopaquinone->Dopachrome->Melanin

Tyrosinase (monophenol dihydroxyl phenylalanine:oxygen oxidoreductase EC 1.14.18.1) participates in this sequence of reactions by especially catalyzing the reaction for conversion of tyrosine into dopa (dihydroxyphenylalanine) and the reaction for conversion of dopa into dopaquinone.

The upper part of the hair follicle appears as a tubular invagination of the epidermis, which is buried just down to the deep layers of the dermis. The lower part, or hair bulb, itself comprises an invagination in which is found the dermal papilla. Around the dermal papilla, in the lower part of the bulb, is an area populated with cells with a high degree of proliferation (matrix cells). These cells are the precursors of the keratinized cells that will constitute the hair. The cells that result from the proliferation of these precursors migrate vertically in the bulb and become gradually keratinized in the upper part of the bulb; this assembly of keratinized cells will form the hair stem. Pigmentation of the hair and of other bodily hairs requires the presence of melanocytes in the bulb of the hair follicle. These melanocytes are in an active state, i.e., they synthesize melanins (or melanin pigments). These pigments are transferred to the keratinocytes intended to form the hair stem, which will give rise to the growth of a pigmented head hair or other bodily hair. This structure is known as a “follicular pigmentation unit”.

It is known that, in the majority of populations, a brown skin coloration and maintenance of a constant coloration of head hair are important aspirations.

It is accepted that the appearance of grey or white bodily hairs and/or head hairs, or canities, is associated with a decrease in melanin in the hair stem. This phenomenon occurs naturally during the life of an individual. However, individuals are seeking to have a more youthful appearance and, with an aesthetic goal, they are often tempted to combat this phenomenon, especially when it occurs at a relatively early age.

Many solutions have thus been proposed in the field of artificial coloration by providing exogenous dyes intended to give the hair a coloration as close as possible to that which it has naturally. Another approach consists in stimulating the natural pigmentation pathway.

Among the proposed solutions, exemplary are compositions containing a phosphodiesterase inhibitor (WO 95/17161), DNA fragments (WO 95/01773), diacylglycerol (WO 94/04122), prostaglandins (WO 95/11003) or pyrimidine 3-oxide derivatives (EP-829,260).

The assignee hereof has now found, unexpectedly, that it is possible to stimulate the synthesis of melanin by the melanocytes by specifically inhibiting the degradation of the prostaglandins synthesized by these melanocytes or those present in its environment.

The involvement of certain prostaglandins in the pigmentation of bodily hairs or the skin in man or animals is described in Wand M., 1997, Arch. Ophtalmol., 115; Abdel Malek et al., 1987, Cancer Res., 47. However, since prostaglandins are molecules with a very short biological half-life and as a result of the local and labile nature of their metabolism (Narumiya S. et al.), it appears important to be able to prolong the activity of the prostaglandins involved in the pigmentation of human skin, bodily hairs and/or head hair.

In WO 04/073 594, the assignee hereof has shown that 15-hydroxyprostaglandin dehydrogenase (15-PGDH) is also expressed in the hair melanocyte, which has never been demonstrated hitherto. In addition, the assignee hereof has demonstrated the presence of 15-PGDH in the dermal papilla and the melanocyte of head hairs, and has proposed administering a 15-PGDH inhibitor to promote the pigmentation of human skin, bodily hairs and/or head hair. It is now possible to locally regulate the level of prostaglandins and especially that present in the melanocyte, in particular of head hair, by acting on the degradation catalyzed both by the 15-PGDH of the melanocyte and of the fibroblast of the dermal papilla.

Type-1 15-PGDH is a key enzyme in the deactivation of prostaglandins, in particular of PGF2-α and PGE2, which are important mediators of the growth and survival of the hair. It corresponds to the classification EC 1.1.1.141 and is NAD⁺-dependent. This enzyme catalyses an oxidation reaction of the hydroxyl on carbon 15 to ketone. It has been isolated from pig kidney; its inhibition has especially been observed with a thyroid hormone, triiodothyronine, at doses very much higher than the physiological doses. Type-2 15-PGDH is itself NADP-dependent.

In this same patent application, the assignee hereof also showed that the hair melanocytes express prostaglandin H synthase 1 (PGHS-1 or COX-1, E.C.:1.14.99.1). This demonstrates for the first time that the hair melanocytes have an autonomous prostaglandin metabolism.

In WO 04/073 594, it has moreover been shown that it is possible to specifically inhibit the 15-PGDH present in the dermal papilla and/or in the hair melanocyte. Such an inhibition thus makes it possible to stop the deactivation of the prostaglandins in the environment of the hair melanocyte. The prostaglandins can thus continue, via an autocrine or paracrine route, to stimulate the melanocytes. In point of fact, the application of such inhibitors stimulates the production of melanin by the melanocytes.

According hereto, the term “15-PGDH inhibitor” means any substance, a simple or complex compound, of natural or synthetic origin, capable of inhibiting or reducing the activity of the enzyme 15-PGDH, and/or capable of inhibiting, reducing or slowing down the reaction catalyzed by this enzyme. The 15-PGDH inhibitors according to the present invention are preferably inhibitors of type-1 15-PGDH.

Advantageously, the inhibitor is a specific inhibitor of the NAD-dependent type-1 15-PGDH.

### SUMMARY OF THE INVENTION

Thus, surprisingly, it has now been found that certain 2-oxyacetamide compounds or a salt and/or solvate thereof of formula (I) more fully described hereinbelow, are inhibitors of 15-hydroxyprostaglandin dehydrogenase, in particular of type 1. It has moreover also been found that these same 2-oxyacetamide compounds of formula (I) promote and/or induce and/or stimulate the pigmentation of keratin materials, and also limit the depigmentation and/or bleaching thereof.

The present invention thus features the cosmetic administration of at least one 2-oxyacetamide compound of formula (I) or a salt and/or solvate thereof, as an agent for promoting and/or inducing and/or stimulating the pigmentation of keratin materials and/or as an agent for preventing and/or limiting the depigmentation and/or bleaching of keratin materials and, more particularly, of human keratin fibers, for instance human hair, beard hair, moustache hair, eyelashes and eyebrows.

The present invention more particularly features the cosmetic administration of at least one 2-oxyacetamide compound of formula (I), or a salt and/or solvate thereof, as an agent for preventing and/or limiting the canities of human keratin fibers.

This invention also features the cosmetic formulation of at least one 2-oxyacetamide compound of formula (I), or a salt and/or solvate thereof, into a care and/or makeup composition for inducing and/or stimulating the pigmentation of keratin materials and/or for limiting the depigmentation and/or bleaching thereof, and more particularly of human keratin fibers such as human hair, beard hair, moustache hair, eyelashes and eyebrows.

The present invention more particularly features the cosmetic formulation of at least one 2-oxyacetamide compound of formula (I), or of a salt and/or solvate thereof, into a care and/or makeup composition for preventing and/or limiting the canities of human keratin fibers.

This invention also features the cosmetic formulation of at least one 2-oxyacetamide compound of formula (I), or a salt and/or solvate thereof, into compositions for inducing and/or stimulating the pigmentation of keratin materials and/or for limiting the depigmentation and/or bleaching thereof, and more particularly of human keratin fibers such as human hair, beard hair, moustache hair, eyelashes and eyebrows.

The present invention more particularly features the formulation of at least one 2-oxyacetamide compound of formula (I), or a salt and/or solvate thereof, into compositions for preventing and/or limiting the canities of human keratin fibers such as human hair, beard hair, moustache hair, eyelashes and eyebrows.

The present invention also features a cosmetic process, whether regime or regimen, for inducing and/or stimulating the pigmentation of keratin materials and more particularly of human keratin fibers, and/or for limiting the depigmentation and/or bleaching thereof, comprising applying to said keratin materials an effective amount of at least one 2-oxyacetamide compound of formula (I), or a salt and/or solvate thereof.

This invention also features a regime or regimen for treating the canities of human keratin fibers, in particular the hair, beard hair, moustache hair, the eyelashes and/or the eyebrows, comprising applying to said fibers an effective amount of at least one 2-oxyacetamide compound of formula (I), or a salt and/or solvate thereof.

The 2-oxyacetamide compounds or the salts and/or solvates thereof according to the invention correspond to the general formula (I):

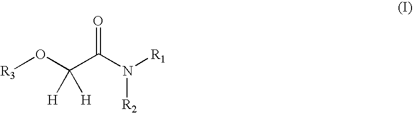

in which:
a) R₁ and R₂ are independently selected from among:

1) a hydrogen, with R₁, different from R₂,

2) a C₁-C₂₀ alkyl radical optionally substituted with at least one substituent A₁

3) a hydrocarbon-based ring C¹ of 3 to 7 atoms optionally fused to at least one ring C² of 4 to 7 atoms, the ring C² optionally containing at least one heteroatom to form a heterocycle Hy₂, these rings C¹ and C² optionally comprising a carbonyl or thiocarbonyl function and/or being substituted with at least one substituent A₂,

4) a heterocycle Hy₁ optionally fused to a ring C² of 4 to 7 atoms, the ring C² optionally containing at least one heteroatom to form a heterocycle Hy₂, these rings Hy₁ and C² optionally comprising a carbonyl or thiocarbonyl function and/or being substituted with at least one substituent A₁,

5) one of the groups C(═NR)R′, C(═NR)NR′R″, COR, CSR, COOR, CONRR′, SO₂R or SO₂NRR′;

b) R₃ is selected from among:

1) a C₁-C₂₀ alkyl radical optionally substituted with at least one substituent A₁,

2) a hydrocarbon-based ring C³ of 3 to 7 atoms optionally fused to at least one ring C⁴ of 4 to 7 atoms optionally containing at least one heteroatom to form a heterocycle Hy₄, these rings C³ and C⁴ optionally comprising a carbonyl or thiocarbonyl function and being substituted with at least one substituent A₂,

3) a heterocycle Hy₃ selected from among pyrrole, furan, thiophene and pyrazole rings and optionally fused to a ring C⁵ representing a phenyl, a pyridine or a pyrimidine, the heterocycle Hy₃ and the ring C⁵ being optionally substituted with at least one substituent A₁,

4) a heterocycle Hy₅ different from Hy₃ and optionally fused to a ring C⁶ of 4 to 7 atoms, these rings Hy₅ and C⁶ optionally comprising a carbonyl or thiocarbonyl function and/or being substituted with at least one substituent A₁, this ring C⁶ optionally containing at least one heteroatom to form a heterocycle Hy₆;

c) R, R′ and R″ are independently selected from among:

1) a hydrogen,

2) a C₁-C₂₀ alkyl radical optionally substituted with at least one substituent A₁,

3) a ring C⁷ of 4 to 7 atoms optionally containing at least one heteroatom to form a heterocycle Hy₇ and/or being optionally fused to a ring C⁸ of 4 to 7 atoms optionally containing at least one heteroatom to form a heterocycle Hy₈, the rings C⁷ and C⁸ being optionally substituted with at least one substituent A₁ and optionally comprising a carbonyl or thiocarbonyl function;

d) A₁ is selected from among:

1) a halogen,

2) a C₁-C₂₀ alkyl radical optionally substituted with a group OR₅,

3) a ring C⁹ of 4 to 15 atoms optionally containing at least one heteroatom to form a heterocycle Hy₉ and/or being optionally fused to a ring C¹⁰ of 4 to 7 atoms, these rings C⁹ and C¹⁰ optionally comprising a carbonyl or thiocarbonyl function and/or at least one substituent A₃ selected from among OR₅, CF₃, a halogen and a C₁-C₂₀ alkyl radical,

4) one of the groups CF₃, CN, OR₄, SR₄, NR₄R′₄, NR₄C(═NR′₄)NR″₄R′₄, COR₄, CSR₄, COOR₄, CONR₄R′₄, NR₄COR′₄, NR₄CONR′₄R′₄, SO₂NR₄R′₄, NR₄SO₂R′₄, SO₂R₄, SiR₄R′₄R″₄, Si(OR₄)(OR′₄)OR″₄ and SO₃H, in which R₄, R′₄, R″₄ and R′₄ independently are each a hydrogen or a C₁-C₂₀ alkyl radical optionally substituted with a heterocycle Hy₁₀ or a group CONR₅R′₅;

e) A₂ is selected from among:

1) a halogen,

2) one of the groups CF₃, CN, OR₄, SR₄, NR₄R′₄, OCOR₄, COR₄, CSR₄, COOR₄, SO₂R₄, SiR₄R′R″₄, NO₂ and OCF₃, in which R₄, R′₄ and R″₄ independently are each a hydrogen or a C₁-C₂₀ alkyl radical optionally substituted with a phenyl radical,

3) a C₁-C₂₀ alkyl radical optionally substituted with a group OR₅,

4) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function and/or at least one substituent A₃ selected from among OR₅, CF₃, a halogen and a C₁-C₂₀ alkyl radical;

f) R₅ and R′₅ are independently selected from among:

1) a hydrogen,

2) a C₁-C₂₀ alkyl radical;

g) Hy₁, Hy₂, Hy₄ to Hy₈, Hy₁₀ and Hy₁₁ independently are each a heterocycle of 4 to 7 atoms optionally having from 1 to 4 heteroatoms selected from N, O and S and combinations thereof and/or comprising a carbonyl or thiocarbonyl function,
h) Hy₉ is a heterocycle of 4 to 15 atoms optionally having from 1 to 5 heteroatoms selected from N, O and S and combinations thereof and/or comprising a carbonyl or thiocarbonyl function.

### DETAILED DESCRIPTION OF BEST MODE AND SPECIFIC/PREFERRED EMBODIMENTS OF THE INVENTION

The compounds of formula (I) according to the invention are specific inhibitors of 15-PGDH; the term “specific inhibitor” means an active agent that has little or no inhibitory activity on the synthesis of prostaglandins, in particular on the synthesis of PGF2-α or PGE2. According to one particular embodiment of the invention, the 15-PGDH inhibitor has little or no inhibitory activity on the synthesis of prostaglandins, especially on the synthesis of PGF2-α or PGE2. In particular, the type-1 15-PGDH inhibitor has little or no inhibitory activity on prostaglandin synthase (abbreviated as PGF synthase or PGFS).

Advantageously, the compounds of formula (I), in salified or non-salified, and solvated or non-solvated form, have inhibitory activity on 15-PGDH that is higher than the inhibitory activity on PGF synthase. These compounds are referred to as selective inhibitors of 15-PGDH relative to PGF synthase. In particular, the ratio from the inhibitory activities of PGF synthase and of 15-PGDH, respectively, for a given concentration, determined especially by means of the concentration that inhibits 50% of the enzymatic activity of PGF synthase (IC_{50fs}) relative to the concentration that inhibits 50% of the enzymatic activity of 15-PGDH (IC_{50dh}), is at least greater than 1, especially at least 3:1 and advantageously greater than or equal to 5:1.

In the text hereinbelow, unless specifically indicated, the term “compound of formula (I)” means not only the compound of formula (I) but also one of its salts or solvates, in particular hydrates, or one of its solvated salts (in particular hydrated salts).

According to the invention, the term “salts of a compound of formula (I)” means the organic or mineral salts of a compound of formula (I).

As mineral salts according to the invention, exemplary are the sodium or potassium salts and also the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts; hydroxides, hydrohalides (for example hydrochlorides), carbonates, hydrogen carbonates, sulfates, hydrogen phosphates and phosphates.

The organic salts according to the invention are, for example, the triethanolamine, monoethanolamine, diethanolamine, hexadecylamine and N,N,N′,N′-tetrakis(2-hydroxypropyl)ethylenediamine salts, and also those of organic acids, for instance citrates, lactates, glycolates, gluconates, acetates, propionates, fumarates, oxalates and tartrates.

As possible solvates of the compounds of formula (I), exemplary are the hydrates, alkoxides and hydroalkoxides.

According to the invention, the compounds of formula (I) are in isolated, i.e., non-polymeric, form.

According to the invention, the term “at least one” means one or more (2, 3 or more).

In particular, the compositions may contain one or more compounds of formula (I). This or these compound(s) may be cis or trans or Z or E isomers or a mixture of cis/trans or Z/E isomers. They may also be in tautomeric form. This or these compound(s) may be enantiomers and/or diastereoisomers or a mixture of these isomers, in particular a racemic mixture.

For the purposes of the invention, the term “hydrocarbon-based” refers to a group of hydrogen and carbon atoms.

For the purposes of the invention, the term “alkyl radical” means a hydrocarbon-based radical, which may be linear or branched and saturated or unsaturated. In particular, the alkyl radical contains from 1 to 20 and preferably from 1 to 10 carbon atoms. Examples of alkyl radicals according to the invention include methyl, ethyl, isopropyl, n-butyl, tert-butyl, n-hexyl, 2-ethylhexyl, ethylene and propylene radicals.

As halogen atoms according to the invention, exemplary are chlorine, fluorine and bromine atoms, and preferably chlorine and fluorine atoms.

The rings C¹ to C⁴ and C⁶ to C¹⁴ and also the heterocycles Hy₁, Hy₂ and Hy₄ to Hy₁₁ may be saturated or unsaturated. They in particular comprise 5 to 6 atoms. According to one particular embodiment of the invention, the ring C⁹ may comprise up to 15 atoms and may represent, for example, a crown ether containing 5 —CH₂CH₂O— units. In addition, the rings C¹, C³, C⁷, C⁹, C¹¹, C¹³, Hy₁, Hy₃, Hy₅, Hy₇, Hy₉ and Hy₁₁ may be fused to one or more other rings of identical or different chemical nature.

Exemplary saturated hydrocarbon-based rings according to the invention include cyclopropyl, cyclobutyl, cyclopentyl, cyclohexyl and cycloheptyl radicals. Unsaturated hydrocarbon-based rings include cyclohexenyl and phenyl radicals. Fused hydrocarbon-based rings according to the invention include naphthyl and azulenyl radicals. Fused rings of different nature according to the invention include benzofuran, dibenzofuran, benzothiophene, benzothiazole, indole, benzimidazole, quinoline, isoquinoline, quinazoline, carboline, chromene, carbazole and fluorene radicals.

According to the invention, R₁ and/or R₂ and/or R₃ are each a hydrocarbon-based ring as defined above, but also a heterocycle comprising from 1 to 4 heteroatoms selected from N, O and S, and combinations thereof. In addition, these hydrocarbon-based or heterocyclic rings optionally comprise a carbonyl or thiocarbonyl function.

As examples of rings containing a carbonyl or thiocarbonyl function according to the invention, the following rings are representative:

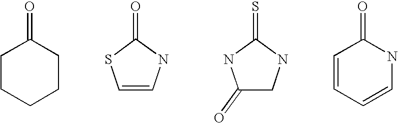

According to one particular embodiment of the invention, R₁ and/or R₂ is a heterocycle Hy₁ selected especially from among azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, dihydropyrazole, pyrazolidine, oxazole, dihydrooxazole, oxazolidine, isoxazole, dihydroisoxazole isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine, diazepine. In particular, Hy₁ is a γ-butyrolactone, a piperidine or a 1,3-benzodioxole optionally substituted with a linear or branched, and especially saturated, C₁-C₄ alkyl radical.

Advantageously, R₁ is hydrogen and R₂ is one of the following groups:

a linear or branched saturated C₁-C₁₀ alkyl radical, for instance a methyl, ethyl, butyl, propyl, pentyl or hexyl radical, optionally substituted with one or two substituents A₁,

a saturated hydrocarbon-based ring of 3 to 6 carbon atoms,

a saturated or unsaturated heterocycle Hy₁ of 5, 6 or 7 atoms, comprising 1 or 2 heteroatoms selected from O, N and S and optionally a carbonyl function and/or from 1 to 4 substituents A′₂,

a phenyl ring optionally substituted with one or two substituents A″₂ selected from among a halogen (especially fluorine or chlorine), NO₂, OCF₃, CF₃, OR₄, OCH₂R₄, COOR₄, a C₁-C₁₀ alkyl radical that is especially saturated (for instance ethyl, propyl, tert-butyl, hexyl or methyl), and a hydrocarbon-based or heterocyclic aromatic ring,

a phenyl ring fused to one or two saturated or unsaturated hydrocarbon-based or heterocyclic rings of 5 to 6 atoms, thus forming fused rings, for instance indane, carbazole, benzodioxole, fluorene or dibenzofuran rings.

According to the invention, the substituents A₁ or A₂ borne by the same ring or the same alkyl radical may be identical or different. In addition, they may be identical or different for R₁, R₂ and R₃. Similarly, the radicals R₄, R′₄, R″₄ and R′₄ may be identical or different for the same group, the same radical or from a substituent A₁ to a substituent A₂.

In particular, A₁ is a hydrocarbon-based ring or a heterocycle comprising 1 or 2 heteroatoms selected from O and N, this hydrocarbon-based or heterocyclic ring comprising 5 to 6 atoms and optionally a carbonyl function and/or a substituent A₃; or a group selected from SiR₄R′₄R″₄, COOR₄, NR₄R′₄, OR₄, SR₄ and CONR₄R′₄.

According to one particular embodiment of the invention, R₄, R′₄, R″₄ and R′₄ are each a hydrogen, a phenyl radical or a C₁-C₁₀ alkyl radical, for instance a methyl, ethyl, tert-butyl, n-butyl, n-propyl, isopropyl or pentyl radical.

Advantageously, A′₂ is a linear or branched and especially saturated C₁-C₁₀ alkyl radical such as methyl.

According to one particular embodiment of the invention, A₃ is a linear or branched and especially saturated C₁-C₁₀ alkyl radical such as methyl, CF₃, a halogen atom, for instance F, OH or OCH₃.

According to one particular embodiment of the invention, R₃ is a heterocycle Hy₅ selected from among azetidine, dihydropyrrole, pyrrolidine, dihydrofuran, tetrahydrofuran, dihydrothiophene, tetrahydrothiophene, imidazole, dihydroimidazole, imidazolidine, thiazole, dihydrothiazole, thiazolidine, dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine.

Advantageously, R₃ is one of the following groups:

a saturated or unsaturated hydrocarbon-based ring, optionally substituted with one or more substituents A₂ selected from among a phenyl radical, COOR₄, OR₄, COR₄, CN, a saturated or unsaturated heterocycle containing 5 or 6 atoms and comprising 1 or 2 heteroatoms selected from O, S and N, for instance pyrrole or imidazole, a halogen (fluorine or chlorine), a phenyl radical optionally substituted with CN, a linear or branched and especially saturated C₁-C₁₀ alkyl radical, for instance ethyl, methyl, isopropyl, isobutyl or tert-butyl, with R₄ representing a hydrogen or a linear or branched and especially saturated C₁-C₁₀ alkyl radical, for instance methyl or ethyl,

a phenyl ring fused to one or two saturated or unsaturated hydrocarbon-based or heterocyclic rings C⁴ of 5 to 6 atoms, the ring(s) C⁴ optionally comprising a carbonyl function and/or optionally being substituted with a linear or branched and especially saturated C₁-C₁₀ alkyl radical, for instance methyl or ethyl, this phenyl ring fused to this or these ring(s) C⁴ thus forming fused rings such as indane, benzothiazole, quinoline, carbazole, fluorene, fluorenone or dibenzofuran rings,

a linear or branched and especially saturated C₁-C₁₀ alkyl radical, for instance methyl, optionally substituted with one or two substituents A₁, for instance a phenyl radical.

As examples of heterocycles Hy₂, Hy₄, Hy₆, Hy₇, Hy₈, Hy₉, Hy₁₀ and Hy₁₁ according to the invention, independently representative are azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine rings or a 15-C-5 crown ether for Hy₉. Preferably, pyrrole, pyrrolidine, imidazole, furan, thiophene, oxazole, thiazole, isoxazole, isothiazole, oxadiazole, pyrazole, tetrazole, pyridine, pyrimidine, triazole, pyrazine, pyridazine, piperidine, piperazine or morpholine rings or a 15-C-5 crown ether for Hy₉.

According to one preferred embodiment of the invention, the 2-oxyacetamide compounds have formula (1a) below, or a salt and/or a solvate thereof:

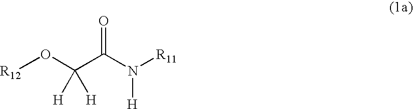

in which:
α) R₁₁ is selected from among:

1) a linear or branched and especially saturated C₁-C₁₀ alkyl radical optionally substituted with at least one substituent A4,

2) a saturated or unsaturated hydrocarbon-based ring C¹⁵ of 5 to 6 atoms optionally substituted with at least one substituent A₅;

β) R₁₂ is selected from among:

1) a linear or branched and especially saturated C₁-C₁₀ alkyl radical optionally substituted with at least one substituent A₄,

2) a saturated or unsaturated hydrocarbon-based ring C¹⁶ of 5 to 6 atoms, optionally fused to at least one saturated or unsaturated ring C¹⁷ of 5 to 6 atoms optionally containing at least one heteroatom to form a heterocycle Hy₁₇, these rings C¹⁶ and C¹⁷ optionally comprising a carbonyl function and/or being substituted with at least one substituent A₅;

γ) A4 is selected from among:

1) a saturated or unsaturated ring C¹⁸ of 5 to 6 atoms optionally containing at least one heteroatom to form a heterocycle Hy₁₈, this ring or this heterocycle being optionally substituted with at least one substituent A₆ selected from among OR₁₄, CF₃, a halogen, for instance fluorine or chlorine, and a linear or branched and especially saturated C₁-C₁₀ alkyl radical;

2) one of the groups CF₃, CN, OR₁₃, SR₁₃, NR₁₃R′₁₃, NR₁₃C(═NR′₁₃)NR″₁₃R′₁₃, COR₁₃, COOR₁₃, CONR₁₃R′₁₃, NR₁₃COR′₁₃, NR₁₃CONR′₁₃R″₁₃, SO₂NR₁₃R′₁₃, NR₁₃SO₂R′₁₃, SO₂R₁₃, SiR₁₃R′₁₃R″₁₃ and SO₃H, in which R₁₃, R′₁₃, R″₁₃ and R′₁₃ independently are each a hydrogen or a linear or branched and especially saturated C₁-C₁₀ alkyl radical;

δ) A₅ is selected from among:

1) a halogen, for instance fluorine or chlorine,

2) one of the groups CF₃, CN, OR₁₅, SR₁₅, NR₁₅R′₁₅, OCOR₁₅, COR₁₅, COOR₁₅, SO₂R₁₅, SiR₁₅R′₁₅R″₁₅, NO₂ and OCF₃, in which R₁₅, R′₁₅ and R″₁₅ independently are each a hydrogen or a linear or branched and especially saturated C₁-C₁₀ alkyl radical optionally substituted with a phenyl radical,

3) a linear or branched and especially saturated C₁-C₁₀ alkyl radical optionally substituted with a group OR₁₄,

4) a saturated or unsaturated ring C¹⁹ of 5 to 6 atoms optionally containing at least one heteroatom to form a heterocycle Hy₁₉ and/or possibly comprising a carbonyl function;

ε) R₁₄ is selected from among:

1) a hydrogen,

2) a linear or branched and especially saturated C₁-C₁₀ alkyl radical;

η) Hy₁₇, Hy₁₈ and Hy₁₉ independently are each a heterocycle of 5 to 6 atoms possibly having from 1 to 4 heteroatoms selected from N, O and S and combinations thereof and/or comprising a carbonyl function.

According to one preferred embodiment of the invention, the 2-oxyacetamide compounds have one of the formulae (II) to (V) below, or a corresponding salt and/or solvate thereof:

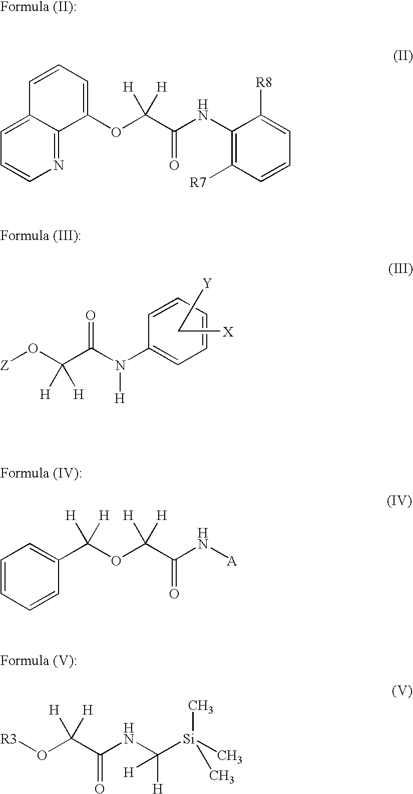

in which:
(i) R7 and R8 independently represent:

1) a hydrogen

2) a halogen F or Cl,

3) one of the groups CF₃, CN, OR₄, SR₄, NHR₆, NR₆R′₆, OCOR₄, COR₄, CSR₄, COOR₄, SO₂R₄, SiR₄R′₄R″₄ and OCF₃, in which R₄, R′₄ and R″₄ independently are each a hydrogen or a C₁-C₂₀ alkyl radical optionally substituted with a phenyl radical and in which R₆ and R₁₆ are each a C₁-C₂₀ alkyl radical optionally substituted with a phenyl radical,

4) a C₁-C₂₀ alkyl radical optionally substituted with a group OR₅ in which R₅ is a hydrogen or a C₁-C₂₀ alkyl radical,

5) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² possibly comprising a carbonyl or thiocarbonyl function;

(ii) Z is one of the following rings and heterocycles:

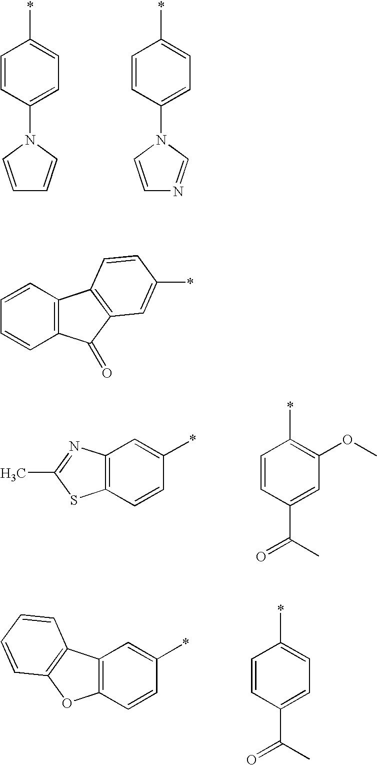

(iii) X and Y independently are each a hydrogen or A₂ as defined above for formula (I) or may form a fused ring C¹³ of 4 to 7 atoms and preferably of 5 to 6 atoms, optionally comprising at least one heteroatom selected from N, O and S and combinations thereof, and being optionally substituted with one or more substituents A₂, and optionally fused to another ring C¹⁴; with the proviso that:

when Z is the dibenzofuran heterocycle, X and Y independently represent:

1) a hydrogen,

2) a halogen (F or Cl),

3) one of the groups CF₃, CN, OR₉, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently are each a hydrogen or a C₁-C₂₀ alkyl radical optionally substituted with a phenyl radical and in which R₉ is a hydrogen or a C₂-C₂₀ alkyl radical optionally substituted with a phenyl radical,

4) a C₂-C₂₀ alkyl radical optionally substituted with a group OR₅,

1) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function; or

when Z is the ring:

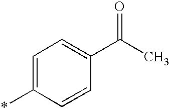

X and Y independently represent:

1) hydrogen, in this case X is different from Y,

2) fluorine,

3) one of the groups OR₁₀, SR₄, NR₄R′₄, OCOR₄, COR₄, CSR₄, COOR₆, SiR₄R′₄R″₄ and OCF₃, in which R₄, R′₄ and R″₄ independently are each a hydrogen or a C₁-C₂₀ alkyl radical optionally substituted with a phenyl radical, in which R₆ is a C₁-C₂₀ alkyl radical optionally substituted with a phenyl radical and in which R₁₀ is a hydrogen atom or a benzyl radical or a C₃-C₂₀ alkyl radical optionally substituted with a phenyl radical,

4) a C₂-C₂₀ alkyl radical optionally substituted with a group OR₅,

5) X and Y may also form a saturated or unsaturated, 5-membered (or 5-atom) ring C¹³ optionally containing one or two oxygen atoms and being optionally fused to another saturated or unsaturated ring C¹⁴;

(iv) A is one of the following three groups:

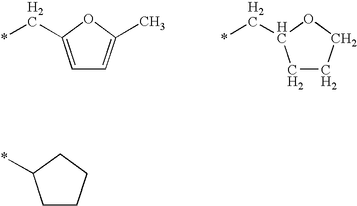

(v) R₃ has the same definition as for formula (I).

Moreover, the rings C¹¹ and C¹² have the same meaning as above.

The effective amount of a compound of formula (I) (salified or non-salified, and solvated or non-solvated) corresponds to the amount of compound required to obtain the desired result (i.e., to induce, stimulate keratin pigmentation and especially of the hair and the eyelashes and/or to reduce their depigmentation and/or their bleaching). One skilled in this art is thus capable of evaluating this effective amount, which depends on the nature of the compound used, the individual on whom it is applied and the time of this application.

In the text hereinbelow, and unless otherwise indicated, the amounts of the various ingredients in the composition are given as weight percentages relative to the total weight of the composition.

To provide an order of magnitude, according to the invention, the compound of formula (I) (salified or non-salified, and solvated or non-solvated) or a mixture of compounds of formula (I) (salified or non-salified, and solvated or non-solvated) may be used in an amount ranging from 10⁻³% to 10% of the total weight of the composition, preferably in an amount representing from 10⁻³% to 5% to more preferably from 10⁻²% to 2% of the total weight of the composition, for example from 0.5% to 2%.

The compositions of the invention may be for cosmetic or pharmaceutical use. The compositions of the invention are preferably for cosmetic use. In addition, the composition must contain a non-toxic, physiologically acceptable medium that can be applied to human skin, including the scalp and the eyelids and to keratin fibers. For the purposes of the invention, the term “cosmetic” means a composition of pleasant appearance, odor and feel.

The compounds of formula (I) (salified or non-salified, and solvated or non-solvated) may be used in compositions that should be ingested, injected or applied to the skin or to keratin fibers (to any area of skin or fibers to be treated).

According to the invention, the compound of formula (I) or a mixture of compounds of formula (I) may be administered orally in an amount of from 0.1 to 300 mg per day, for example from 5 to 10 mg/day.

A preferred composition of the invention is a composition for cosmetic use and in particular for topical application to the skin and keratin fibers, and more especially to the scalp, the hair and the eyelashes.

This compositions may be in any known presentation form that is suitable for the mode of administration.

For topical application to the skin or keratin fibers, the compositions may be in the form of an aqueous, alcoholic or aqueous-alcoholic solution or suspension, or an oily suspension or solution, an emulsion or dispersion of more or less fluid consistency and especially of liquid or semi-liquid consistency, obtained by dispersion of a fatty phase in an aqueous phase (O/W) or conversely (W/O), a solid (O/W) or (W/O) emulsion or dispersion, a more or less fluid or solid aqueous, aqueous-alcoholic or oily gel, a free or compacted powder to be used in unmodified form or to be incorporated into a physiologically acceptable medium, or alternatively microcapsules, microparticles or vesicular dispersions of ionic and/or nonionic type.

Compositions in the form of a foam or alternatively in the form of a spray or aerosol, then comprising a pressurized propellant, are also intended.

Same may also be in the form of a lotion, serum, milk, O/W or W/O cream, gel, unguent, ointment, powder, balm, patch, impregnated pad, cake or foam.

In particular, the compositions for application to the scalp or the hair may be in the form of a haircare lotion, for example for daily or twice-weekly application, a shampoo or a hair conditioner, in particular for twice-weekly or weekly application, a liquid or solid scalp cleansing soap for daily application, a hairstyle shaping product (lacquer, hair setting product or styling gel), a treatment mask, a foaming gel or cream for cleansing the hair. Same may also be in the form of a hair dye or mascara to be applied with a brush or a comb.

Moreover, for application to the eyelashes or body hairs, the compositions of the invention may be in the form of a pigmented or unpigmented mascara, to be applied with a brush to the eyelashes or alternatively to beard or moustache hair.

For a composition for administration by injection, same may be in the form of an aqueous lotion or an oily suspension. For oral administration, the composition may be in the form of capsules, granules, drinkable syrups or tablets.

According to one particular embodiment, the composition according to the invention are in the form of a hair cream or hair lotion, a shampoo, a conditioner for the hair or a mascara for the hair or for the eyelashes.

The amounts of the various constituents of the physiological medium of the composition according to the invention are those generally employed in the fields under consideration. In addition, these compositions are formulated according to the usual methods.

When the composition is an emulsion, the proportion of the fatty phase may range from 2% to 80% by weight and preferably from 5% to 50% by weight relative to the total weight of the composition. The aqueous phase is adjusted as a function of the content of fatty phase and of compound(s) (I) and also of that of the optional additional ingredients, to obtain 100% by weight. In practice, the aqueous phase is from 5% to 99.9% by weight.

The fatty phase may contain fatty or oily compounds that are liquid at room temperature (25° C.) and atmospheric pressure (760 mmHg), which are generally known as oils. These oils may be mutually compatible or incompatible and may form a macroscopically homogeneous liquid fatty phase or a two-phase or three-phase system.

In addition to the oils, the fatty phase may contain waxes, gums, lipophilic polymers or “pasty” or viscous products containing solid fractions and liquid fractions.

The aqueous phase contains water and optionally an ingredient that is miscible in all proportions with water, for instance C₁ to C₈ lower alcohols such as ethanol or isopropanol, polyols, for instance propylene glycol, glycerol or sorbitol, or alternatively acetone or ether.

For a composition in emulsion form, the composition may contain one or more emulsifiers optionally combined with one or more co-emulsifiers used to obtain a composition in emulsion form, these emulsifiers and co-emulsifiers being those generally employed in cosmetics and pharmaceuticals. Their nature also depends on the sense of the emulsion. In practice, the emulsifier and, where appropriate, the co-emulsifier are present in the composition in a proportion ranging from 0.1% to 30% by weight, preferably from 0.5% to 20% by weight and better still from 1% to 8% by weight. The emulsion may also contain microcapsules or microparticles, and vesicular dispersions and especially lipid vesicles and especially liposomes.

When the composition is in the form of an oily solution or gel, the fatty phase may constitute more than 90% of the total weight of the composition.

Advantageously, for a hair application, the compositions of the invention are aqueous, alcoholic or aqueous-alcoholic solution or suspension and preferably a water/ethanol solution or suspension. The alcoholic fraction may constitute from 5% to 99.9% to better still from 8% to 80%.

For a mascara application, the compositions of the invention are especially in the form of a wax-in-water or wax-in-oil dispersion, a gelled oil or an aqueous gel, which may be pigmented or unpigmented.

The compositions of the invention may also comprise other additional ingredients usually employed in the fields under consideration, selected from among aqueous-phase or oily-phase solvents, thickeners or gelling agents, dyes that are soluble in the medium of the composition, solid particles such as fillers or pigments, antioxidants, sequestrants, preservatives, fragrances, electrolytes, neutralizers, film-forming polymers, UV blockers, for instance sunscreens, cosmetic and pharmaceutical active agents with a beneficial effect on the skin or keratin fibers, other than the compounds of formula (I), and mixtures thereof. These additives may be present in the composition in the amounts generally included in cosmetics and dermatology, and especially in a proportion of from 0.01% to 50% to preferably from 0.1% to 20%, for example from 0.1% to 10%, relative to the total weight of the composition. Depending on their nature, these additives may be introduced into the fatty phase, into the aqueous phase and/or into the lipid vesicles and especially liposomes.

Needless to say, one skilled in this art will take care to select the optional additional ingredients and/or the amount thereof such that the advantageous properties of the compositions according to the invention, i.e., the inhibition of 15-PGDH and in particular the increase in the density of keratin fibers and/or the reduction in their bleaching, are not, or are not substantially, adversely affected by the envisaged addition.

As solvents according to the invention, exemplary are C₂ to C₈ lower alcohols, for instance ethanol, isopropanol, propylene glycol and certain light cosmetic oils, for instance C₆ to C₁₆ alkanes.

Exemplary oils according to the invention include oils of mineral origin (liquid petroleum jelly or hydrogenated isoparaffin), oils of plant origin (liquid fraction of shea butter, sunflower oil, apricot oil, fatty alcohol or fatty acid), oils of animal origin (perhydrosqualene), synthetic oils (fatty acid ester, purcellin oil), silicone oils (linear or cyclic polydimethylsiloxane, phenyl trimethicone) and fluoro oils (perfluoropolyethers). Waxes that are exemplary include silicone waxes, beeswax, rice wax, candelilla wax, carnauba wax, paraffin wax and polyethylene wax.

Exemplary emulsifiers according to the invention include glyceryl stearate, glyceryl laurate, sorbitol stearate, sorbitol oleate, alkyl dimethicone copolyols (with alkyl≧8) and mixtures thereof for a W/O emulsion. Polyethylene glycol monostearate or monolaurate, polyoxyethylenated sorbitol stearate or oleate, and dimethicone copolyols, and mixtures thereof, may also be included in an O/W emulsion.

Exemplary hydrophilic gelling agents according to the invention, include carboxyvinyl polymers (carbomer), acrylic copolymers such as acrylate/alkylacrylate copolymers, polyacrylamides, polysaccharides such as hydroxypropylcellulose, natural gums and clays, and exemplary lipophilic gelling agents are modified clays, for instance Bentones, metal salts of fatty acids, for instance aluminum stearates, hydrophobic-treated silica and ethylcellulose, and mixtures thereof.

Exemplary cosmetic or pharmaceutical active agents other than the compounds of formula (I) include hydrophilic active agents selected from among proteins or protein hydrolysates, amino acids, polyols, urea, allantoin, sugars and sugar derivatives, water-soluble vitamins, plant extracts (those from Iridacea plants or from soybean) and hydroxy acids such as fruit acids or salicylic acid; and lipophilic active agents such as retinol (vitamin A) and derivatives thereof especially an ester (retinyl palmitate), tocopherol (vitamin E) and derivatives thereof especially an ester (tocopheryl acetate), essential fatty acids, ceramides, essential oils, salicylic acid derivatives, for instance 5-n-octanoylsalicylic acid, hydroxy acid esters, and phospholipids, for instance lecithin, and mixtures thereof.

Advantageously, the compositions according to the invention also comprise at least one prostaglandin or one prostaglandin derivative, for instance prostaglandins of the series 2 especially including PGF2-α and PGE2 in salt or ester form (for example the isopropyl esters), derivatives thereof, for instance 16,16-dimethyl PGE2, 17-phenyl PGE2, 16,16-dimethyl PGF2-α, 17-phenyl PGF2-α, prostaglandins of the series 1, for instance 11-deoxyprostaglandin E1, 1-deoxyprostaglandin E1 in salt or ester form, analogues thereof, especially latanoprost, (5E)-7-{(1R,2R,3R,5S)-3,5-dihydroxy-2-[(3R)-3-hydroxy-5-phenylpentyl]cyclopentyl}hept-5-enoic acid, viprostol, bimatoprost, cloprostenol, travoprost, fluprostenol, cloprostenol, butaprost, unoprostone and misoprostol, and salts or esters thereof.

The subject compositions may also comprise at least the compound of formula (I), salified or non-salified, solvated or non-solvated, encapsulated in particular in liposomal form, as described especially in WO 94/22468. Thus, the compound encapsulated may be delivered selectively to the hair follicle.

The compositions according to the invention may be topically applied to the areas of the skin to be treated and in particular to the alopecic areas of the scalp and the hair of an individual, or only to the white hairs, and optionally left in contact for several hours and optionally rinsed off.

The compositions containing an effective amount of a compound of formula (I), salified or non-salified, solvated or non-solvated, may, for example, be applied in the evening, kept in contact throughout the night and optionally shampooed out in the morning. These applications may be repeated daily for one or more months according to the individual.

Advantageously, in the regime or regimen according to the invention, from 5 μL and 10 ml of a solution or composition as defined above, comprising from 0.001% to 5% of 15-PGDH inhibitor, are topically applied to the areas of the scalp and/or the hair to be cared for or treated.

In order to further illustrate the present invention and the advantages thereof, the following specific examples are given, it being understood that same are intended only as illustrative and in nowise limitative. In said examples to follow, all parts and percentages are given by weight, unless otherwise indicated.

### EXAMPLES

Examples of 2-oxyacetamide compounds of formula (II) according to the invention include the following compounds:

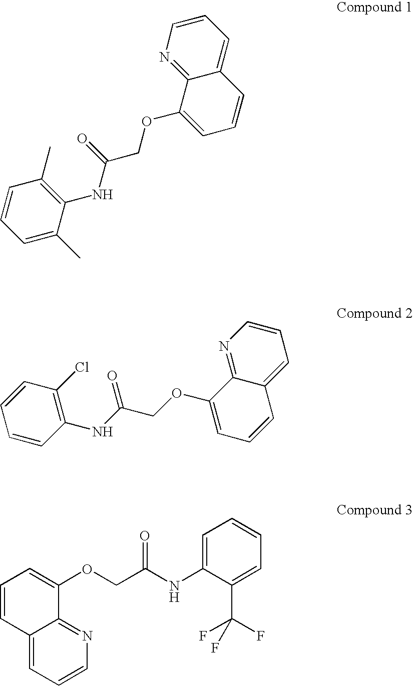

Examples of 2-oxyacetamide compounds of formula (III) according to the invention include the following compounds:

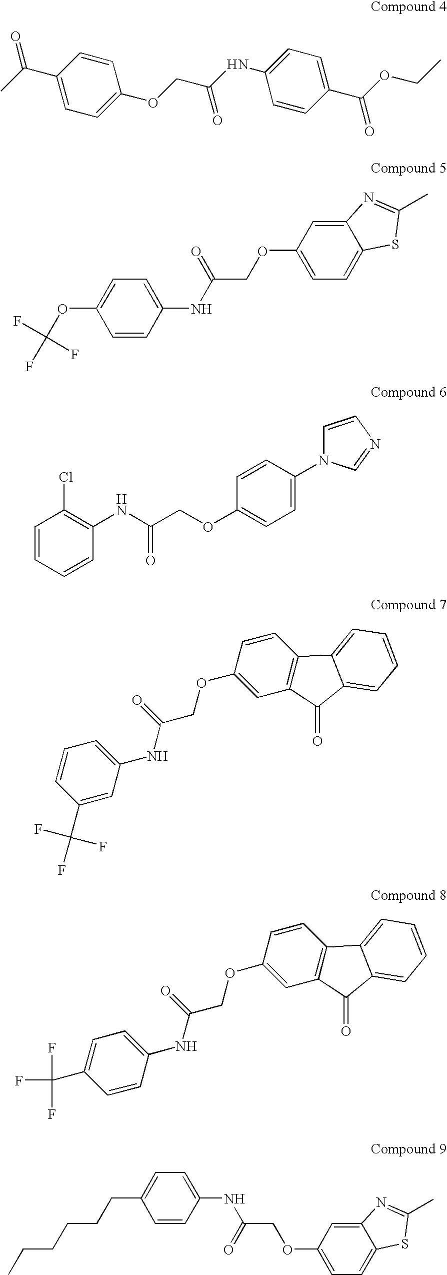

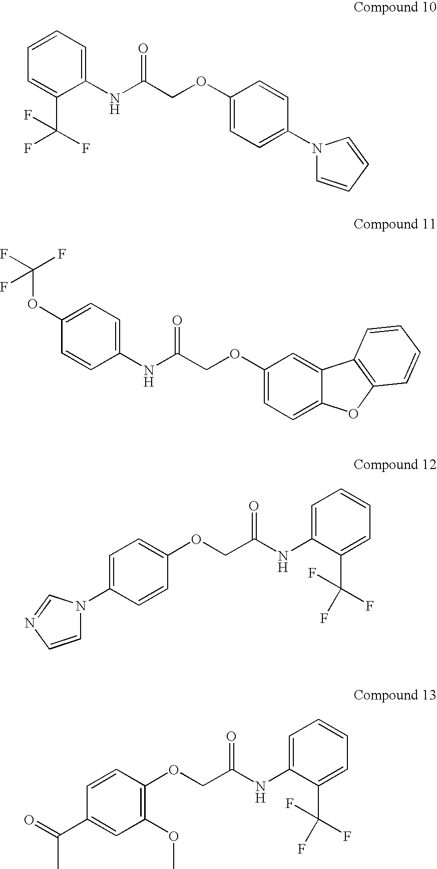

Examples of 2-oxyacetamide compounds of formula (IV) according to the invention include the following compounds:

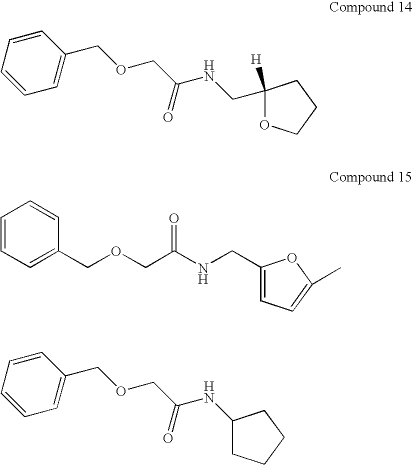

Examples of 2-oxyacetamide compounds of formula (V) according to the invention include the following compounds:

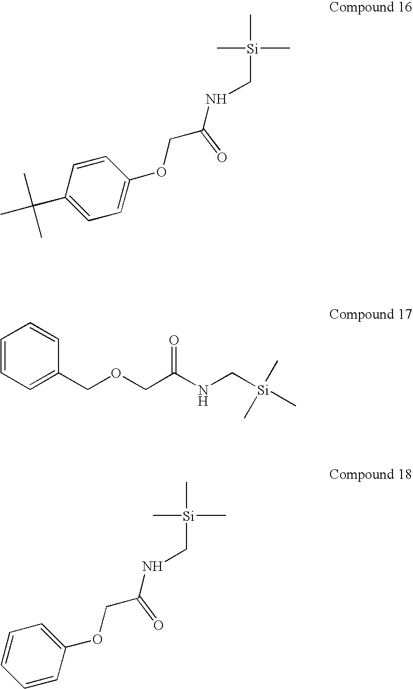

According to another embodiment of the invention, the 2-oxyacetamide compounds have one of the following three formulae:

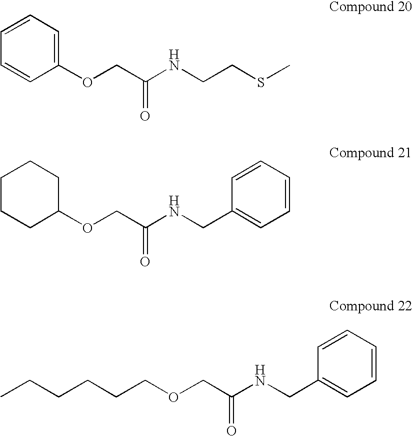

The compounds of formula (I) according to the invention, whether they are novel or known, may be synthesized according to the following reaction scheme:

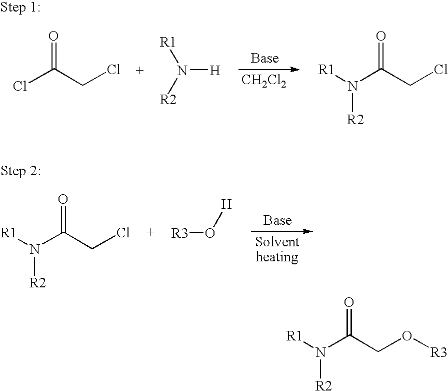

The operating conditions (solvent, base, temperature) for the synthesis of the compounds of formula (I) depend on the starting reagents.

As other examples of 2-oxyacetamide compounds of formula (I) according to the invention are the following compounds:

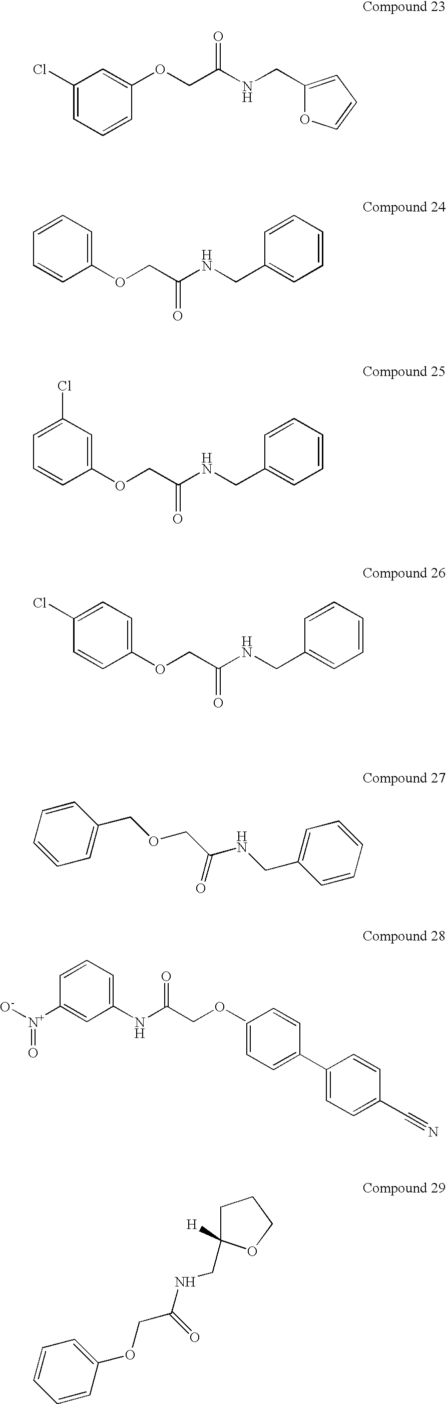

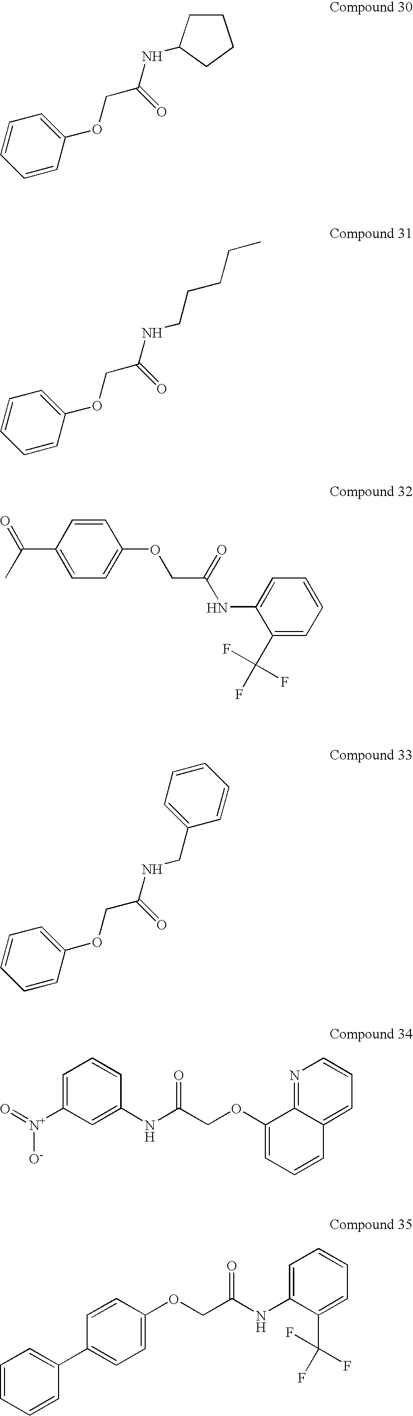

Advantageously, the compounds according to the invention are compounds 2, 3, 5, 6, 8, 9, 10, 12, 13, 18, 19, 21, 23, 24, 28, 32, 34 and 35 and in particular compounds 2, 3, 5, 6, 8, 9, 10, 12, 13, 18, 19 and 21, and compounds 23 and 24. Preferably, the compounds of the invention are compounds 2, 3, 12, 13 and 21 and also compounds 23 and 24.

Examples of synthesis of compounds in accordance with the invention will now be given as non-limiting illustrations.

### Example 1

### Reaction Scheme for the Synthesis of Compound 21

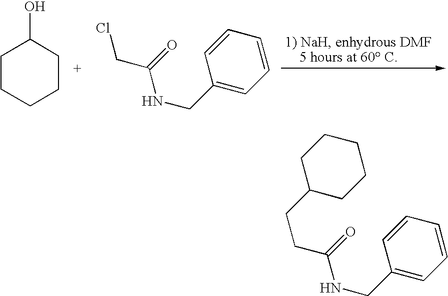

Procedure:

0.63 ml of freshly distilled cyclohexanol (6 mmol) are introduced into a three-necked flask under a flow of argon and diluted in 10 ml of anhydrous dimethylformamide. 60% sodium hydride (0.24 g; 6 mmol) is added portionwise to the reaction medium. The reaction mixture is then stirred at room temperature for 1 hour and a solution of N-benzyl-2-chloroacetamide (1 g; 5.44 mmol) in 10 ml of anhydrous DMF is then added. The medium is maintained at a temperature of 60° C. for 5 hours. The reaction mixture is concentrated to the maximum and then diluted with 100 ml of dichloromethane. The organic phase is washed with water (twice 50 ml) and then with saturated sodium chloride solution. The organic phase is dried over sodium sulfate, filtered and then concentrated to the maximum. The crude product is taken up in a minimum amount of ethyl ether and stirred for 30 minutes. The solid is removed and the filtrate is purified on silica gel (eluent: dichloromethane). After distilling off the solvent and drying under vacuum in the presence of phosphorous pentoxide, a beige-colored solid (0.36 g, in a yield (yld) of 24%) is obtained.

The analyses of the product (Nuclear Magnetic Resonance and Mass Spectrum) are in accordance with formula 21.

### Example 2

### Reaction Scheme for the Synthesis of Compound 22

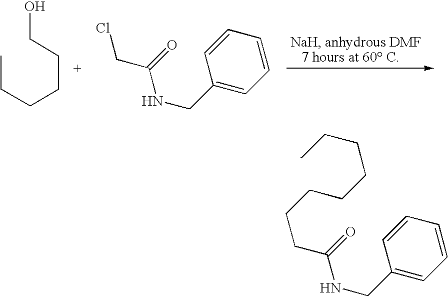

Procedure:

0.7 ml of anhydrous hexanol (5.44 mmol) is diluted in 20 ml of anhydrous DMF in a 50 ml three-necked flask equipped with a condenser and a thermometer, and under a flow of nitrogen. 60% sodium hydride (0.24 g; 6 mmol) is added portionwise to the reaction medium. After addition, the reaction medium is stirred at room temperature for 2 hours and a solution of N-benzyl-2-chloroacetamide (1 g; 5.44 mmol) in 10 ml of anhydrous DMF is then added. The medium is maintained at a temperature of 60° C. for 7 hours. The reaction mixture is concentrated to the maximum and then diluted with 100 ml of dichloromethane. The organic phase is washed with water (twice 50 ml) and then with saturated sodium chloride solution. The organic phase is dried over sodium sulfate, filtered and then concentrated to the maximum. The crude product is taken up in a minimum amount of ethyl ether and stirred for 30 minutes. The solid is removed and the filtrate is purified on silica gel (eluent: dichloromethane). After evaporating off the solvent and drying under vacuum, a yellow oil is obtained (18.5 mg, yld=2%).

The analyses of the product (NMR and Mass Spectrum) are in accordance with those expected for compound 22.

### Example 3

### Reaction Scheme for the Synthesis of compound 23

Compound 23 is prepared in two steps.

### Step 1: Synthesis of the Chloroacetamide (a)

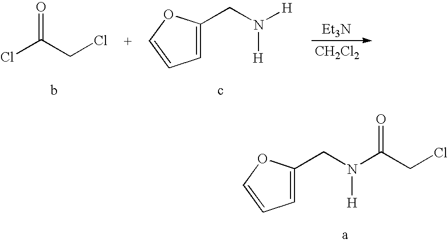

40 ml of furfurylamine (c), 500 ml of dichloromethane and 69.9 ml of triethylamine (abbreviated to Et₃N) are introduced into a three-necked flask. After cooling the reaction medium to 10° C., 37.1 ml of chloroacetyl chloride (b) diluted in 100 ml of dichloromethane are added dropwise while maintaining the temperature below 15° C. The reaction medium is then stirred for 3 hours at room temperature. Water is then added. The organic phase is washed with water (twice 100 ml), then with 1N dilute hydrochloric acid solution (twice 50 ml), again with water and then with saturated sodium chloride solution. The organic phase is washed over sodium sulfate and then filtered and concentrated to give 66 g of a brown solid (compound a, yield=84%).

### Analysis of Product (a)

Mass Spectrometry (MS): the quasi-molecular ions (MH)⁺, (MNa)⁺ of the expected molecule C₇H₈ClNO₂ are mainly detected.

Nuclear Magnetic Resonance (NMR): ¹H (DMSO, 400 MHz) δ ppm: 8.66 (1H, s broad, NH); 7.58 (1H, dd, Hₐᵣ); 6.40 (1H, dd Hₐᵣ); 6.27 (1H, dd, Hₐᵣ); 4.30 (2H, d, CH₂); 4.09 (2H, s, CH₂).

broad s=broad singlet
d=doublet
dd=doubled doublet

These results are in accordance with the formula of compound (a).

### Step 2: Synthesis of Compound 23 ((2-(3-chlorophenoxy)-N-furan-2-ylmethylacetamide

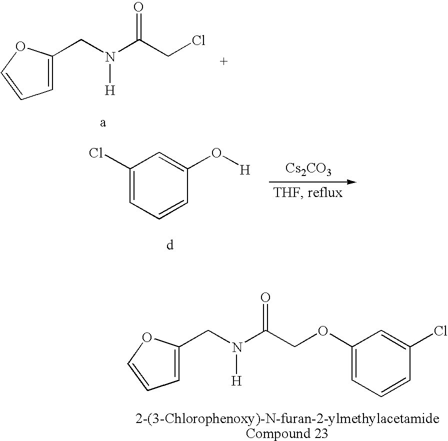

1 g of 3-chlorophenol (d), 20 ml of tetrahydrofuran (THF) and 1.9 g of cesium carbonate are introduced into a three-necked flask. A solution 1.34 g of compound (a) prepared above in 20 ml of dichloromethane (DCM) is then added. The organic medium is refluxed for 5 hours; water is then added. The organic phase is washed with water (twice 100 ml), with 1N dilute sodium hydroxide solution (twice 50 ml), again with water (twice 100 ml) and then with saturated sodium chloride solution. The organic phase is dried over sodium sulfate and then filtered and concentrated to give 1.5 g of a light-brown solid (compound 23, yield=73%).

### Analysis of the Product Obtained:

NMR: ¹H (DMSO, 400 MHz) δ ppm: 8.59 (1H, t, NH); 7.56 (1H, bs, H—Ar); 7.32 (1H, m H—Ar); 7.04 (1H, d, H—Ar); 7.02 (1H, dd, H—Ar); 6.94 (1H, dd, H—Ar); 6.38 (1H, m, H—Ar); 6.21 (1H, m, H—Ar); 4.57 (2H, s, CH₂); 4.33 (2H, d, CH₂).

s=singlet
broad s=broad singlet
d=doublet
dd=doubled doublet
t=triplet
m=multiplet
H—Ar=aromatic hydrogen

Mass Spectrometry: the quasi-molecular ion (MH)⁺ of the expected molecule C₁₃H₁₂ClNO₃ is mainly detected.

These results are in accordance with the formula of compound 23.

### Examples 4, 5 and 6

### Reaction Scheme for the Synthesis of Compounds 24, 25 and 26

These compounds were prepared according to the same process as for compound 21, replacing the cyclohexane with, respectively, phenol for compound 24, 3-chlorophenol for compound 25, and 4-chlorophenol for compound 26.

The mass spectra (+ and − ESU) are in accordance with the expected structures.

### Examples 7, 8 and 9

### Reaction Scheme for the Synthesis of Compounds 1, 2 and 3 of General Formula (II)

Reaction Scheme for the Synthesis of Compound 1:

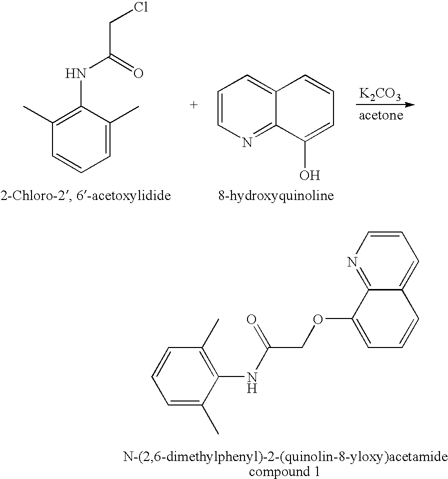

Procedure:

A suspension of 28 mg of potassium carbonate in 1 ml of acetone is introduced into a reaction tube equipped with a magnetic bar. A solution of 6.4 mg of 8-hydroxyquinoline (CAS: 148-24-3; Fluka reference: F55080) in 0.5 ml of acetone and a solution of 7.9 mg of 2-chloro-2′,6′-acetoxylidide (CAS: 1131-01-7) in 0.5 ml of acetone are then added. The medium is diluted with 1 ml of acetone and the tube is stoppered and refluxed for 24 hours. The reaction medium is cooled to room temperature and filtered through a sinter funnel, and the solid is rinsed with 2.5 ml of acetone. The filtrate is concentrated under vacuum, taken up in 300 μl of a 9/1 dichloromethane/methanol mixture and added to a suspension of 80 mg of PL-TBD resin (1,3,4,6,7,8-hexahydro-2H-pyrimido[1,2-a]pyrimidine resin charged at 2.7 mmol/g) in 1.5 ml of dichloromethane. The medium is left for 24 hours and then filtered through a sinter funnel and concentrated. Compound 1 (N-(2,6-dimethylphenyl)-2-(quinolin-8-yloxy)acetamide) is obtained in the form of a brown solid (9.2 mg) in a yield of 75%.

Analysis:

Mass Spectrometry: (+ and − ESI): (M+H)⁺=307 and (M+Na)⁺=330

Compounds 2 and 3 were obtained in a similar manner by reacting 8-hydroxyquinoline (CAS: 148-24-3; Fluka reference: F55080) with the following commercial chloroacetamides according to the same procedure:

N-(2-chlorophenyl)-2-chloroacetamide (3289-76-7) for compound 2 (N-(2-chlorophenyl)-2-quinolin-8-yloxy)acetamide) obtained in the form of a brown solid (7.4 mg) in a yield of 59%.

N-(2-chloroacetyl)-2-(trifluoromethyl)aniline (3792-04-9) for compound 3 (2-(quinolin-8-yloxy)-N-(2-trifluoromethylphenyl)acetamide) obtained in the form of an orange solid (6.5 mg) in a yield of 47%.

### Examples 10 to 19

### Reaction Scheme for the Synthesis of Compounds 4 to 13 of General Formula (III)

Reaction Scheme for the Synthesis of Compound 4:

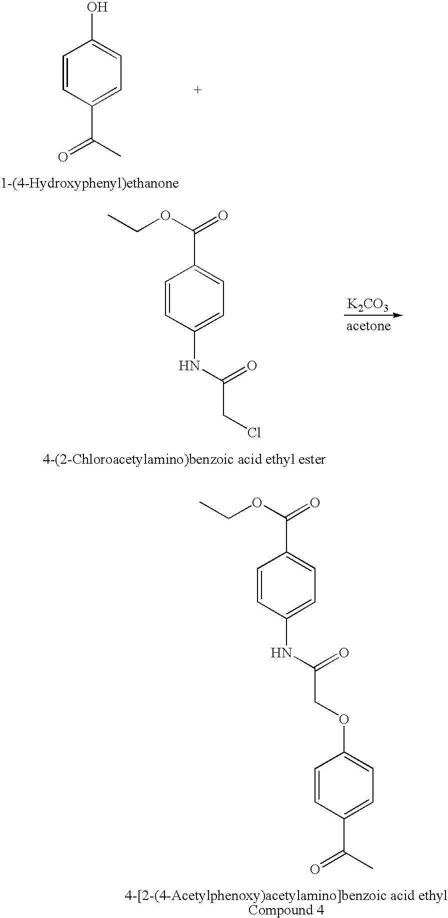

The procedure is the same as that described for the synthesis of the compounds of general formula (II).

Thus, compound 4 is obtained in the form of white crystals (11.2 mg) in a yield of 82% by reaction from 1-(4-hydroxyphenyl)ethanone (4-hydroxyacetophenone CAS: 99-93-4) and ethyl 4-(2-chloroacetamido)benzoate (4-(2-chloroacetylamino)benzoic acid ethyl ester CAS: 26226-72-2).

Similarly, compounds 5, 6, 7, 8, 9, 10, 11, 12 and 13 are obtained according to the table below:

| | Starting | Starting | Com- | | Appear- | | |
|---|---|---|---|---|---|---|---|
| Structure | phenol | chloride | pound | Name | ance | Mass (yield) | |
| 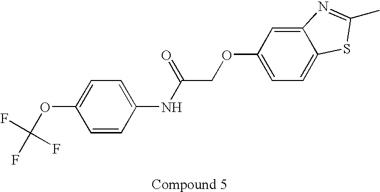 | 2-methyl-5-benzo-thiazolol | N-(chloro-acetyl)-4-(tri-fluoro-methoxy)-aniline | 5 | 2-(2-Methyl-benzothiazol-5-yloxy)-N-(4-trifluoromethoxy-phenyl)acetamide | whitesolid | 5.3 | mg (35%) |
| 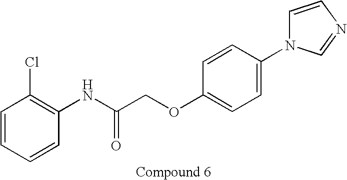 | 4-(Imidazol-1-yl)phenol | N-(2-chloro-phenyl)-2-chloro-acetamide | 6 | N-(2-Chloro-phenyl)-2-(4-imidazol-1-yl-phenoxy)-acetamide | beigesolid | 10.3 | mg (79%) |
| 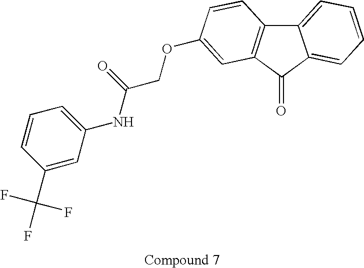 | 2-Hydroxy-9-Fluo-renone | N-(chloro-acetyl)-3-(tri-fluoro-methyl)aniline | 7 | 2-(9-Oxo-9H-fluoren-2-yloxy)-N-(3-trifluoromethyl-phenyl)acetamide | orangesolid | 6.6 | mg (41%) |
| 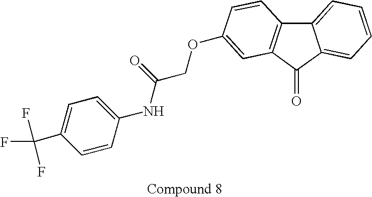 | 2-Hydroxy-9-Fluo-renone | N-chloro-acetyl-4-(tri-fluoro-methyl)aniline | 8 | 2-(9-Oxo-9H-fluoren-2-yloxy)-N-(4-trifluoromethyl-phenyl)acetamide | yellowsolid | 2.4 | mg (15%) |
| 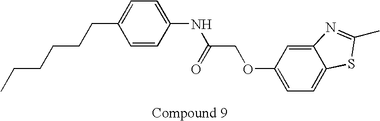 | 2-methyl-5-benzo-thiazolol | | 9 | N-(4-Hexyl-phenyl)-2-(2-methyl-benzo-thiazol-5-yloxy)acetamide | beigesolid | 5.1 | mg (33%) |
| 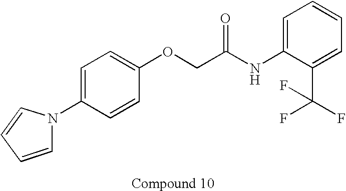 | 4-(1H-Pyrrol-1-yl)phenol | N-(chloro-acetyl)-2-(tri-fluoro-methyl)aniline | 10 | 2-(4-Pyrrol-1-yl-phenoxy)-N-(2-tri-fluoro-methyl-phenyl)acetamide | orangecrystals | 4.7 | mg (33%) |
| 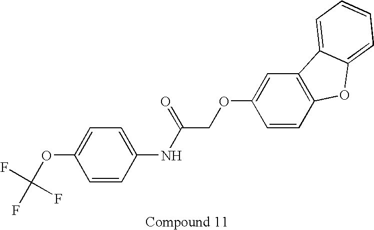 | 2-hydroxydibenzo-furan | N-(chloro-acetyl)-4-(tri-fluoro-methoxy)-aniline | 11 | 2-(Di-benzo-furan-2-yloxy)-N-(4-tri-fluoro-methoxy-phenyl)acetamide | whitesolid | 4.8 | mg (30%) |
| 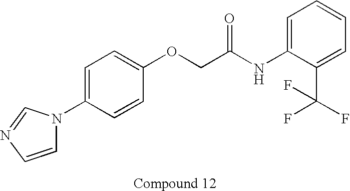 | 4-(Imidazol-1-yl)phenol | N-(chloro-acetyl)-2-(tri-fluoro-methyl)aniline | 12 | 2-(4-Imidazol-1-yl-phenoxy)-N-(2-tri-fluoro-methyl-phenyl)acetamide | ochresolid | 4.9 | mg (34%) |
| 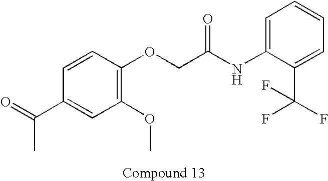 | 4-hydroxy-3-methoxy-aceto-phenone,97% | N-(chloro-acetyl)-2-(tri-fluoro-methyl)aniline | 13 | 2-(4-Acetyl-2-methoxy-phenoxy)-N-(2-tri-fluoro-methyl-phenyl)acetamide | yellowsolid | 7 | mg (48%) |

### Examples 20, 21 and 22

### Reaction Scheme for the Synthesis of Compounds 14, 15 and 16 of General Formula (IV)

Reaction Scheme for the Synthesis of Compound 14:

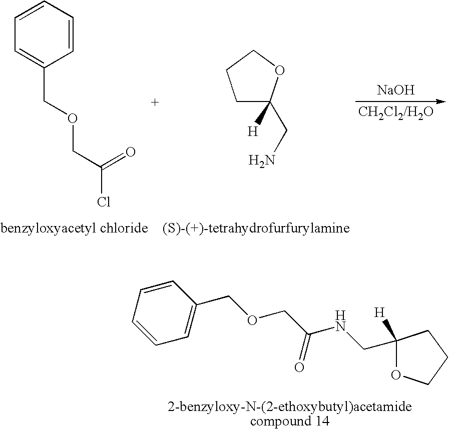

Procedure:

A solution of 9.6 mg of benzyloxyacetyl chloride (CAS: 19810-31-2; Aldrich reference: 30, 101-9) in 0.5 ml of dichloromethane and a solution of 5.2 mg of (S)-(+)-tetrahydrofurfurylamine (CAS: 7175-81-7) in 0.5 ml of 1N sodium hydroxide are introduced into a reaction tube equipped with a magnetic bar. The medium is diluted with 0.5 ml of dichloromethane, and 1 ml of 1N sodium hydroxide solution is then introduced therein. The reaction medium is maintained under magnetic stirring for 24 hours and then thrown onto a liquid-liquid extraction cartridge (Chem Elut Varian) and the organic phase is evaporated. Compound 14 (2-benzyloxy-N-(tetrahydrofuran-2-ylmethyl)acetamide) is obtained in a yield of 72% (7.2 mg).

### Analysis

Mass Spectrometry: ESI (+ and −): (M+H)⁺=250 and (M+Na)⁺=272

Compounds 15 and 16 are prepared according to same procedure. The data are collated in the following table:

| | Starting | | | | | | |
|---|---|---|---|---|---|---|---|
| | acid | | Com- | | | Mass | |
| Structure | chloride | Starting amine | pound | Name | Appearance | (yield) | |
| 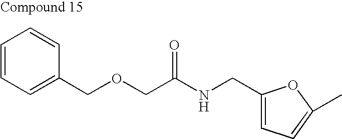 | benzyl-oxyacetylchloride | 5-Methyl-furfuryl-amine | 15 | 2-Benzyloxy-N-(5-methyl-furan-2-ylmethyl)-acetamide | white solid | 7.9 | mg (76%) |
| 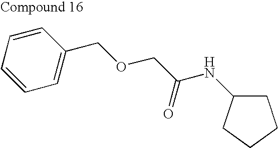 | benzyl-oxyacetylchloride | cyclopentyl-amine | 16 | 2-Benzyloxy-N-cyclopentyl-acetamide | white solid | 8 | mg (86%) |

### Examples 23 to 25

### Reaction Scheme for the Synthesis of Compounds 17, 18 and 19 of General Formula (V)

The procedure is the same as that described for the synthesis of the compounds of general formula (IV).

Compounds 17, 18 and 19 are prepared according to the data collated in the following table.

| | Starting acid | | | | Appear- | Mass |
|---|---|---|---|---|---|---|
| Structure | chloride | Starting amine | Compound | Name | ance | (yield) |
| 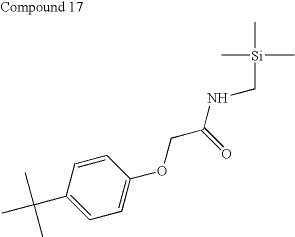 | 4-tert-butyl-phenoxy-acetylchloride | (trimethylsilyl)methyl-amine | 17 | 2-(4-tert-Butyl-phenoxy)-N-trimethylisilanylmethyl-acetamide | whitesolid | 5.0 mg(85%) |
| 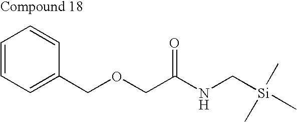 | benzyl-oxyacetylchloride | (trimethylsilyl)methyl-amine | 18 | 2-Benzyloxy-N-trimethylsilanylmethyl-acetamide | whitesolid | 7.5 mg(75%) |
| 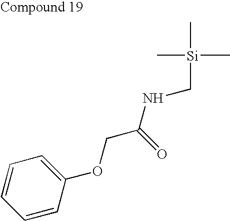 | Phenoxy-acetylchloride | (trimethylsilyl)methyl-amine | 19 | 2-Phenoxy-N-trimethylsilanylmethyl-acetamide | whitesolid | 7.1 mg(75%) |

### Example 26

### Reaction Scheme for the Synthesis of Compound 20

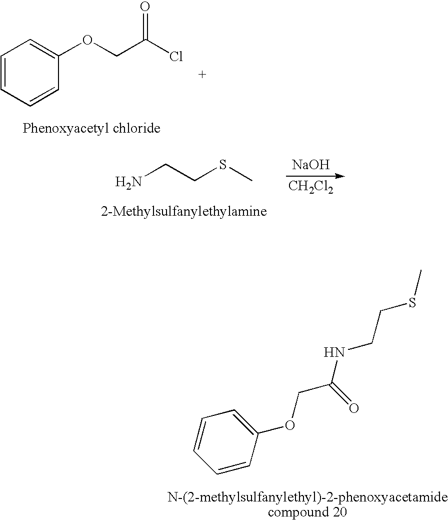

The procedure is the same as that used for the synthesis of the compounds of general formula (IV). Compound 20 is prepared in the form of a white solid (7.8 mg) in a yield of 87%.

### Example 27

### Demonstration of the Specific Inhibitory Properties on 15-PGDH of the Compounds of Formula (I)

Test on Type-1 15-PGDH:

The enzyme 15-PGDH is obtained as described in FR 02/05067 filed by L'Oréal, as a suspension in a medium adapted to a concentration of 0.3 mg/ml and then frozen at −80° C. For the purposes of the test, this suspension is thawed and stored in ice.

Separately, a pH 7.4 100 mM Tris buffer is prepared, containing 0.1 mM of dithiothreitol (D5545, Sigma-Aldrich, L'isle D'Abeau Chesne, BP 701, 38297, Saint Quentin Fallavier), 1.5 mM of β-NAD (N6522, Sigma-Aldrich, L'isle D'Abeau Chesne, BP 701, 38297, Saint Quentin Fallavier), 50 μM of prostaglandin E₂ (P4172, Sigma-Aldrich, L'isle D'Abeau Chesne, BP 701, 38297, Saint Quentin Fallavier).

0.965 ml of this buffer (adjusted to 37° C. beforehand) is introduced into the cuvette of a spectrophotometer (Perkin-Elmer, Lambda 2) thermostatically maintained at 37° C., the measuring wavelength of which is set at 340 nm. 0.035 ml of enzymatic suspension at 37° C. is introduced into the cuvette concomitantly with the recording (corresponding to an increase in the optical density at 340 nm). The maximum reaction rate is recorded.

The test values (containing the compounds of formula (I)) are compared with the control value (without compound (I)); the results indicated represent either the percentage of inhibition of the enzymatic activity of 15-PGDH for a given concentration of compound of formula (I), or the concentration at which the compound of formula (I) reduces by 50% the enzymatic activity of 15-PGDH, i.e., IC_{50dh}.

The results are reported in the table below:

| | | % inhibition of |
|---|---|---|
| | | 15-PGDH at |
| Compound | Structure | 100 μM |
| 1 | 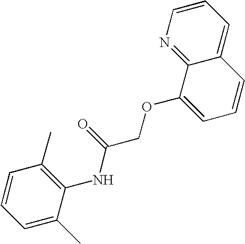 | 49 |
| 2 | 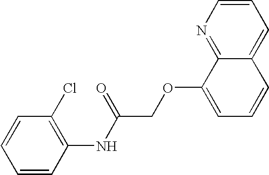 | 115 |
| 3 | 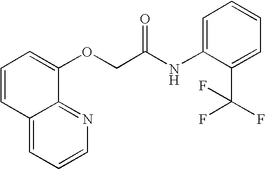 | 128 |
| 4 | 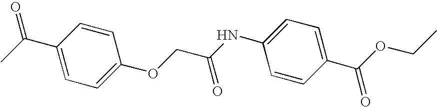 | 49 |
| 5 | 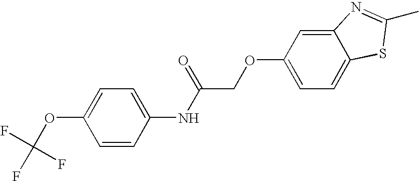 | 53 |
| 6 | 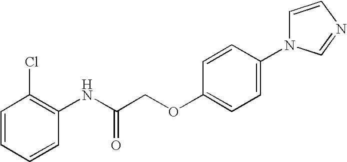 | 54 |
| 7 | 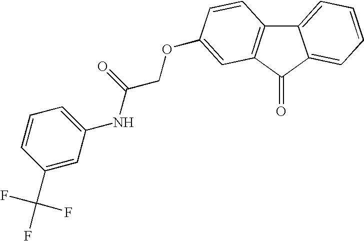 | 61 |
| 8 | 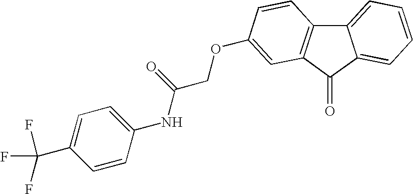 | 65 |
| 9 | 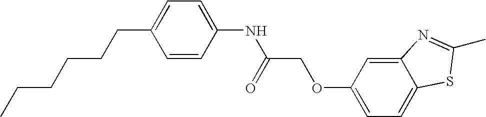 | 69 |
| 10 | 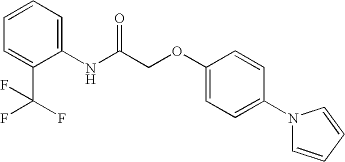 | 71 |
| 11 | 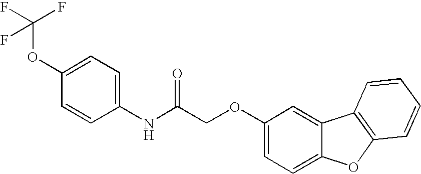 | 74 |
| 12 | 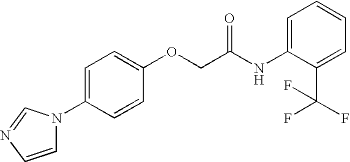 | 125 |
| 13 | 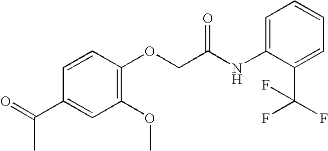 | 126 |
| 14 | 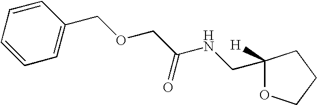 | 50 |
| 15 | 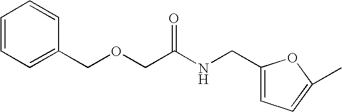 | 51 |
| 16 | 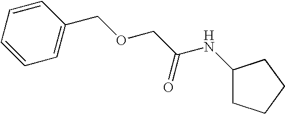 | 76 |
| 17 | 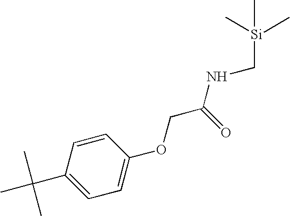 | 50 |
| 18 | 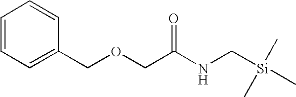 | 88 |
| 19 | 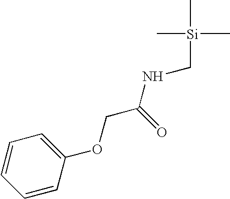 | 95 |
| 20 | 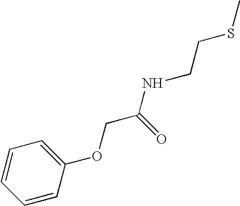 | 85 |
| 27 | 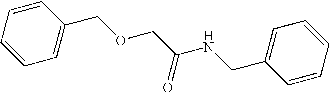 | 48 |
| 28 | 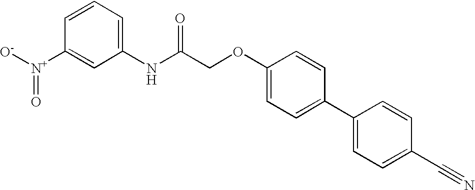 | 54 |
| 29 | 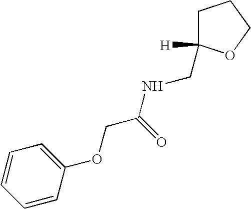 | 54 |
| 30 | 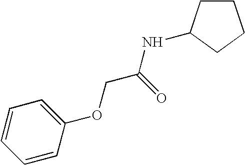 | 56 |
| 31 | 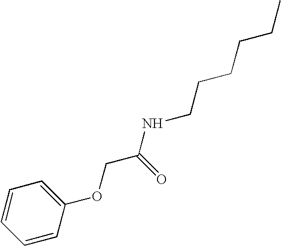 | 57 |
| 32 | 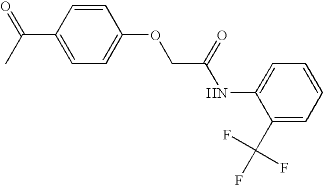 | 59 |
| 33 | 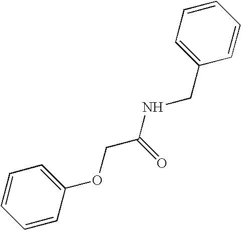 | 61 |
| 34 | 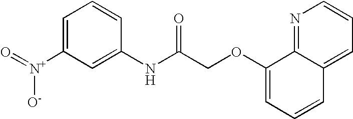 | 69 |
| 35 | 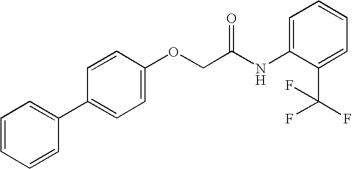 | 70 |

Moreover, compounds 21 to 26 were also tested.

| | | Inhibition of | |
|---|---|---|---|
| | | 15-PGDH | |
| | | | % inhibition |
| | | | at |
| Compound | Structure | IC₅₀ | 50 μM |
| 21 | 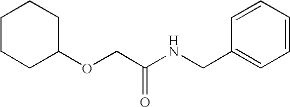 | — | 63 |
| 22 | 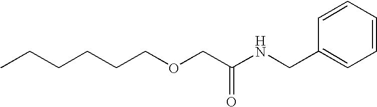 | — | 47 |
| 23 | 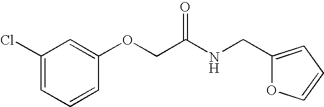 | 16 μM | 80 |
| 24 | 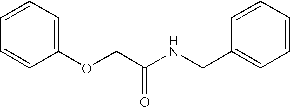 | — | 55 |
| 25 | 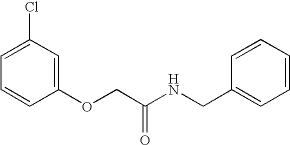 | — | 50 |
| 26 | 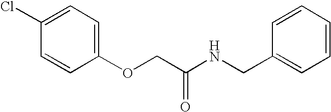 | — | 30 |

From these tables, it is clearly seen that the compounds of formula (I) are inhibitors of type-1 15-PGDH.

The compositions below are obtained via the usual techniques commonly employed in the cosmetic or pharmaceutical field.

### Example 28

### Demonstration of the Effect of Compound 23 on the Tyrosinase Activity of Normal Human Melanocytes in Culture

Melanocyte Culture Conditions:

Normal human epidermal melanocytes are cultured (9th passage) in “melanocytes growth medium M2” supplemented with Promocell C24300 supplements 50 IU/ml of penicillin and 50 μg/ml of streptomycin (Invitrogen) under standard temperature conditions (37° C.) and 5% CO₂.

Preparation of the Solutions of the Test Compound:

The compound is dissolved in absolute ethanol (PGE2) or in DMSO (15-PGDH inhibitors). The final titer of the prepared solutions is 10 mM. The final test concentrations range from 0.1 to 10 μM (PGE2) and 1 to 10 μM (15-PGDH inhibitors).

Principle of the Tests:

The tests performed consist in measuring the inhibitory effect on 15-PGDH alone or combined with PGE2* on the overall activity of tyrosinase.

PGE2 is a pigmentation modulator that is deactivated by the enzyme 15-PGDH.

The melanocytes are cultured in 96-well plates, to the point of confluence (culture conditions described in a), and the supernatant of each of the wells is then removed and replaced with medium containing the compounds to be evaluated, i.e., (in triplicate) the 15-PGDH inhibitor alone at 1 μM and 10 μM or for each of these first conditions in the presence of 0.1 μM, 1 μM or 10 μM of PGE2.

The cultures are maintained under these various conditions for 72 hours, the supernatants are then removed and the cell carpets are washed with a phosphate buffer (pH=7.2). The cells are lysed with a pH 7.2 phosphate buffer containing 1% Triton X100.

The tyrosinase activity of each of the lysates is then estimated against a fungal tyrosinase range (Sigma T7755).

The methodology employed uses L-Dopa as reaction substrate (45 minutes at 37° C.), and the formation of melanin is assessed by measuring the absorbance at 450 nm.

In this test, two references are also evaluated: kojic acid (a tyrosinase inhibitor introduced at 0.1% into the culture medium) and isobutylmethylxanthine (IBMX), a phosphodiesterase inhibitor which, by limiting the catabolism of cAMP, contributes towards increasing the overall tyrosinase activity as assessed in this test.

Results:

It was observed that compound 23 (at 1 μM) has the property of stimulating the activity of tyrosinase in the presence of PGE2 (1 and 10 μM).

The results are as follows:

Compound 23 (1 μM)+PGE2 (1 μM)→+31% stimulation of the tyrosinase activity

Compound 23 (1 μM)+PGE2 (10 μM)→+28% stimulation of the tyrosinase activity

PGE2 alone stimulates the activity of tyrosinase by +9% at 1 μM and by +15% at 10 μM.

Compound 23 does indeed have the property of stimulating the activity of tyrosinase.

### Example 29

### Hair Lotion

| | | |
|---|---|---|
| compound 23 | 1.00 | g |
| propylene glycol | 30.00 | g |
| ethyl alcohol | 40.00 | g |
| water | qs 100.00 | g |

This lotion is applied to the scalp, once or twice a day, at a rate of 1 ml per application, by massaging the scalp gently to make the active agent penetrate. The hair is then air-dried. This lotion makes it possible to prevent and/or reduce the canities of the hair.

### Example 30

### Hair Lotion

| | | |
|---|---|---|
| compound 21 | 1.00 | g |
| propylene glycol | 30.00 | g |
| ethyl alcohol | 40.00 | g |
| water | qs 100.00 | g |

This lotion is applied to the scalp, once or twice a day, at a rate of 1 ml per application, by massaging the scalp gently to make the active agent penetrate. The hair is then air-dried. This lotion makes it possible to prevent and/or reduce the canities of the hair.

### Example 31

### Wax/Water Mascara

| | |
|---|---|
| beeswax | 6.00% |
| paraffin wax | 13.00% |
| hydrogenated jojoba oil | 2.00% |
| water-soluble film-forming polymer | 3.00% |
| triethanolamine stearate | 8.00% |
| compound 23 | 1.00% |
| black pigment | 5.00% |
| preservative | qs |
| water | qs 100.00% |

This mascara is applied to the eyelashes like a standard mascara with a mascara brush. It makes it possible to prevent and/or reduce the canities of the eyelashes.

### Example 32

### Hair Lotion

| | | |
|---|---|---|
| compound 21 | 0.10 | g |
| latanoprost | 0.10 | g |
| propylene glycol | 30.00 | g |
| ethyl alcohol | 40.00 | g |
| water | qs 100.00 | g |

This lotion is applied to the scalp, once or twice a day, at a rate of 1 ml per application, by massaging the scalp gently to make the active agent penetrate. The hair is then air-dried. This lotion makes it possible to prevent and/or reduce the canities of the hair.

### Example 33

### Hair Lotion

| | | |
|---|---|---|
| (5E)-7-{(1R,2R,3R,5S)-3,5-dihydroxy-2-[(3R)-3- | 0.10 | g |
| hydroxy-5-phenylpentyl]cyclopentyl}hept-5- | | |
| enoic acid | | |
| compound 21 | 0.10 | g |
| propylene glycol | 30.00 | g |
| ethyl alcohol | 40.00 | g |
| water | qs 100.00 | g |

This lotion is applied to the scalp, once or twice a day, at a rate of 1 ml per application, by massaging the scalp gently to make the active agent penetrate. The hair is then air-dried. This lotion makes it possible to prevent and/or reduce the canities of the hair.

Each patent, patent application, publication, text and literature article/report cited or indicated herein is hereby expressly incorporated by reference in its entirety.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A regime or regimen for promoting and/or inducing and/or stimulating the pigmentation of human keratin materials and/or for preventing and/or limiting the depigmentation and/or bleaching thereof, comprising administering to an individual in need of such treatment, a this effective amount of at least one 2-oxyacetamide compound of formula (I) below, or a salt and/or solvate thereof: 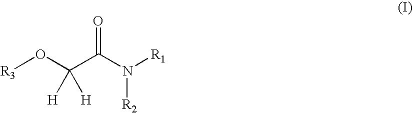
in which:
a) R₁ and R₂ are independently selected from among:
1) a hydrogen, with R₁ different from R₂,
2) a C₁-C₂₀ alkyl radical optionally substituted with at least one substituent A₁
3) a hydrocarbon-based ring C¹ of 3 to 7 atoms optionally fused to at least one ring C² of 4 to 7 atoms, the ring C² optionally containing at least one heteroatom to form a heterocycle Hy₂, these rings C¹ and C² optionally comprising a carbonyl or thiocarbonyl function and/or being substituted with at least one substituent A₂,
4) a heterocycle Hy₁ optionally fused to a ring C² of 4 to 7 atoms, the ring C² optionally containing at least one heteroatom to form a heterocycle Hy₂, these rings Hy₁ and C² optionally comprising a carbonyl or thiocarbonyl function and/or being substituted with at least one substituent A₁,
5) one of the groups C(═NR)R′, C(═NR)NR′R″, COR, CSR, COOR, CONRR′, SO₂R or SO₂NRR′;
b) R₃ is selected from among:
1) a C₁-C₂₀ alkyl radical optionally substituted with at least one substituent A₁,
2) a hydrocarbon-based ring C³ of 3 to 7 atoms optionally fused to at least one ring C⁴ of 4 to 7 atoms optionally containing at least one heteroatom to form a heterocycle Hy₄, these rings C³ and C⁴ optionally comprising a carbonyl or thiocarbonyl function and/or being substituted with at least one substituent A₂,
3) a heterocycle Hy₃ selected from among pyrrole, furan, thiophene and pyrazole rings and/or optionally fused to a ring C⁵ representing a phenyl, a pyridine or a pyrimidine, the heterocycle Hy₃ and the ring C⁵ being optionally substituted with at least one substituent A₁,
4) a heterocycle Hy₅ different from Hy₃ and optionally fused to a ring C⁶ of 4 to 7 atoms, these rings Hy₅ and C⁶ optionally comprising a carbonyl or thiocarbonyl function and/or being substituted with at least one substituent A₁, this ring C⁶ optionally containing at least one heteroatom to form a heterocycle Hy₆;
c) R, R′ and R″ are independently selected from:
1) a hydrogen,
2) a C₁-C₂₀ alkyl radical optionally substituted with at least one substituent A₁,
3) a ring C⁷ of 4 to 7 atoms optionally containing at least one heteroatom to form a heterocycle Hy₇ and/or being optionally fused to a ring C⁸ of 4 to 7 atoms optionally containing at least one heteroatom to form a heterocycle Hy₈, the rings C⁷ and C⁸ being optionally substituted with at least one substituent A₁ and optionally comprising a carbonyl or thiocarbonyl function;
d) A₁ is selected from among:
1) a halogen,
2) a C₁-C₂₀ alkyl radical optionally substituted with a group OR₅,
3) a ring C⁹ of 4 to 15 atoms optionally containing at least one heteroatom to form a heterocycle Hy₉ and/or being optionally fused to a ring C¹⁰ of 4 to 7 atoms, these rings C⁹ and C¹⁰ optionally comprising a carbonyl or thiocarbonyl function and/or at least one substituent A₃ selected from among OR₅, CF₃, a halogen and a C₁-C₂₀ alkyl radical,
4) one of the groups CF₃, CN, OR₄, SR₄, NR₄R′₄, NR₄C(═NR′₄)NR″₄R′₄, COR₄, CSR₄, COOR₄, CONR₄R′₄, NR₄COR′₄, NR₄CONR′₄R″₄, SO₂NR₄R′₄, NR₄SO₂R′₄, SO₂R₄, SiR₄R′₄R″₄, Si(OR₄)(OR′₄)OR″₄ and SO₃H, in which R₄, R′₄, R″₄ and R′₄ independently are each a hydrogen or a C₁-C₂₀ alkyl radical optionally substituted with a heterocycle Hy₁₀ or a group CONR₅R′₅;
e) A₂ is selected from among:
1) a halogen,
2) one of the groups CF₃, CN, OR₄, SR₄, NR₄R′₄, OCOR₄, COR₄, CSR₄, COOR₄, SO₂R₄, SiR₄R′R″₄, NO₂ and OCF₃, in which R₄, R′₄ and R″₄ independently are each a hydrogen or a C₁-C₂₀ alkyl radical optionally substituted with a phenyl radical,
3) a C₁-C₂₀ alkyl radical optionally substituted with a group OR₅,
4) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function and/or at least one substituent A₃ selected from among OR₅, CF₃, a halogen and a C₁-C₂₀ alkyl radical;
f) R₅ and R′₅ are independently selected from among:
1) a hydrogen,
2) a C₁-C₂₀ alkyl radical;
g) Hy₁, Hy₂, Hy₄ to Hy₈, Hy₁₀ and Hy₁₁ independently are each a heterocycle of 4 to 7 atoms optionally having from 1 to 4 heteroatoms selected from N, O and S and combinations thereof and/or comprising a carbonyl or thiocarbonyl function,
h) Hy₉ is a heterocycle of 4 to 15 atoms optionally having from 1 to 5 heteroatoms selected from N, O and S and combinations thereof and/or comprising a carbonyl or thiocarbonyl function, formulated into a cosmetically/pharmaceutically acceptable medium therefor.

2. The regime or regimen as defined by claim 1, wherein formula (I) R₁ is hydrogen and R₂ is one of the following groups:
a saturated C₁-C₁₀ alkyl radical, optionally substituted with one or two substituents A₁,
a saturated hydrocarbon-based ring of 3 to 6 carbon atoms,
a saturated or unsaturated heterocycle Hy₁ of 5, 6 or 7 atoms, comprising 1 or 2 heteroatoms selected from O, N and S and optionally a carbonyl function and/or from 1 to 4 substituents A′₂,
a phenyl ring substituted in particular with one or two substituents A″₂ selected from among a halogen, NO₂, OCF₃, CF₃, OR₄, OCH₂R₄, COOR₄, a C₁-C₁₀ alkyl radical, and a hydrocarbon-based or heterocyclic aromatic ring,
a phenyl ring fused to one or two hydrocarbon-based or heterocyclic rings of 5 to 6 atoms, thus forming fused rings.

3. The regime or regimen as defined by claim 1, wherein formula (I) Hy₁ is a heterocycle selected from among azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, dihydrothiazole, thiazolidine, dihydropyrazole, pyrazolidine, oxazole, dihydrooxazole, oxazolidine, isoxazole, dihydroisoxazole isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine.

4. The regime or regimen as defined by claim 1, wherein formula (I) R₃ is one of the following groups:
a hydrocarbon-based ring, optionally substituted with one or more substituents A′₂ selected from among a phenyl radical, COOR₄, OR₄, COR₄, CN, a heterocycle containing 5 or 6 atoms and comprising 1 or 2 heteroatoms selected from O, S and N, a halogen, a phenyl radical optionally substituted with CN, a linear or branched C₁-C₁₀ alkyl radical wherein R₄ is hydrogen or a C₁-C₁₀ alkyl radical,
a phenyl ring fused to one or two hydrocarbon-based or heterocyclic rings C⁴ of 5 to 6 atoms, the ring(s) C⁴ optionally comprising a carbonyl function and/or optionally being substituted with a saturated C₁-C₁₀ alkyl radical, such phenyl and this or these other ring(s) C⁴ thus forming fused rings,
a saturated C₁-C₁₀ alkyl radical, optionally substituted with one or two substituents A₁.

5. The regime or regimen as defined by claim 1, wherein formula (I) the heterocycles Hy₂, Hy₄, Hy₆, Hy₇, Hy₈, Hy₉, Hy₁₀ and Hy₁₁ independently are each azetidine, pyrrole, dihydropyrrole, pyrrolidine, furan, dihydrofuran, tetrahydrofuran, thiophene, dihydrothiophene, tetrahydrothiophene, imidazole, dihydroimidazole, imidazolidine, thiazole, dihydrothiazole, thiazolidine, pyrazole dihydropyrazole, pyrazolidine, oxazole, dihydrooxazole, oxazolidine, isoxazole, dihydroisoxazole, isoxazolidine, isothiazole, dihydroisothiazole, isothiazolidine, triazole, dihydrotriazole, triazolidine, oxadiazole, dihydrooxadiazole, oxadiazolidine, thiadiazole, dihydrothiadiazole, thiadiazolidine, tetrazole, pyridine, dihydropyridine, tetrahydropyridine, piperidine, pyran, dihydropyran, tetrahydropyran, pyrimidine, dihydropyrimidine, tetrahydropyrimidine, piperazine, pyridazine, pyrazine, triazine, morpholine, azepine and diazepine rings or a 15-C-5 crown ether for Hy₉.

6. The regime or regimen as defined by claim 1, wherein formula (I) the heterocycles Hy₂, Hy₄, Hy₆, Hy₇, Hy₈, Hy₉, Hy₁₀ and Hy₁₁ independently are each pyrrole, pyrrolidine, imidazole, furan, thiophene, oxazole, thiazole, isoxazole, isothiazole, oxadiazole, pyrazole, tetrazole, pyridine, pyrimidine, triazole, pyrazine, pyridazine, piperidine, piperazine or morpholine rings or a 15-C-5 crown ether for Hy₉.

7. The regime or regimen as defined by claim 1, wherein formula (I) A₁ is a hydrocarbon-based ring or a heterocycle comprising 1 or 2 heteroatoms selected from O and N, this hydrocarbon-based or heterocyclic ring comprising 5 to 6 atoms and optionally a carbonyl function and/or a substituent A₃; or a group selected from among SiR₄R′₄R″₄, COOR₄, NR₄R′₄, OR₄, SR₄ and CONR₄R′₄.

8. The regime or regimen as defined by claim 1, wherein formula (I) A₃ is a C₁-C₁₀ alkyl radical, CF₃, a halogen atom, OH or OCH₃.

9. The regime or regimen as defined by claim 1, wherein formula (I) Hy₁ is a γ-butyrolactone, a piperidine or a 1,3-benzodioxole optionally substituted with a C₁-C₄ alkyl radical.

10. The regime or regimen as defined by claim 1, comprising administering a salt of a compound of formula (I) selected from among sodium or potassium salts and also of the ammonium, zinc (Zn²⁺), calcium (Ca²⁺), copper (Cu²⁺), iron (Fe²⁺ and Fe³⁺), strontium (Sr²⁺), magnesium (Mg²⁺) and manganese (Mn²⁺) salts, triethanolamine, monoethanolamine, diethanolamine, hexadecylamine and N,N,N′,N′-tetrakis(2-hydroxypropyl)ethylene diamine salts, hydroxides, hydrohalides, carbonates, hydrogencarbonates, citrates, lactates, glycolates, gluconates, acetates, propionates, fumarates, oxalates, tartrates, sulfates, hydrogen phosphates and phosphates.

11. The regime or regimen as defined by claim 1, comprising administering a hydrate of the 2-oxyacetamide compound of formula (I).

12. The regime or regimen as defined by claim 1, comprising administering at least one 2-oxyacetamide compound of formula (Ia), or a salt and/or solvate thereof: 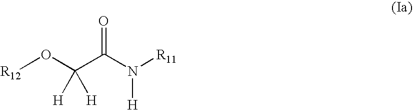
in which:
α) R₁₁ is selected from among:
1) a C₁-C₁₀ alkyl radical optionally substituted with at least one substituent A₄,
2) a hydrocarbon-based ring C¹⁵ of 5 to 6 atoms optionally substituted with at least one substituent A₅;
β) R₁₂ is selected from among:
1) a C₁-C₁₀ alkyl radical optionally substituted with at least one substituent A₄,
2) a hydrocarbon-based ring C¹⁶ of 5 to 6 atoms, optionally fused to at least one ring C¹⁷ of 5 to 6 atoms optionally containing at least one heteroatom to form a heterocycle Hy₁₇, these rings C¹⁶ and C¹⁷ optionally comprising a carbonyl function and/or being substituted with at least one substituent A₅;
γ) A₄ is selected from among:
1) a ring C¹⁸ of 5 to 6 atoms optionally containing at least one heteroatom to form a heterocycle Hy₁₈, this ring or this heterocycle being optionally substituted with at least one substituent A₆ selected from OR₁₄, CF₃, a halogen, and a C₁-C₁₀ alkyl radical;
2) one of the groups CF₃, CN, OR₁₃, SR₁₃, NR₁₃R′₁₃, NR₁₃C(═NR′₁₃)NR″₁₃R′₁₃, COR₁₃, COOR₁₃, CONR₁₃R′₁₃, NR₁₃COR′₁₃, NR₁₃CONR′₁₃R″₁₃, SO₂NR₁₃R′₁₃, NR₁₃SO₂R′₁₃, SO₂R₁₃, SiR₁₃R′₁₃R″₁₃, and SO₃H, in which R₁₃, R′₁₃, R″₁₃ and R′₁₃ independently are each a hydrogen or a C₁-C₁₀ alkyl radical;
δ) A₅ is selected from among:
1) a halogen,
2) one of the groups CF₃, CN, OR₁₅, SR₁₅, NR₁₅R′₁₅, OCOR₁₅, COR₁₅, COOR₁₅, SO₂R₁₅, SiR₁₅R′₁₅R″₁₅, NO₂ and OCF₃, in which R₁₅, R′₁₅ and R″₁₅ independently are each a hydrogen or a C₁-C₁₀ alkyl radical optionally substituted with a phenyl radical,
3) a C₁-C₁₀ alkyl radical optionally substituted with a group OR₁₄,
4) a ring C¹⁹ of 5 to 6 atoms optionally containing at least one heteroatom to form a heterocycle Hy₁₉ and/or optionally comprising a carbonyl function;
ε) R₁₄ is selected from among:
1) a hydrogen,
2) a C₁-C₁₀ alkyl radical;
η) Hy₁₇, Hy₁₈ and Hy₁₉ independently are each a heterocycle of 5 to 6 atoms optionally having from 1 to 4 heteroatoms selected from N, O and S and combinations thereof and/or comprising a carbonyl function.

13. The regime or regimen as defined by claim 1, comprising administering at least one 2-oxyacetamide compound having one of the formulae (II) to (V) below, or one of the corresponding salts and/or solvates thereof: 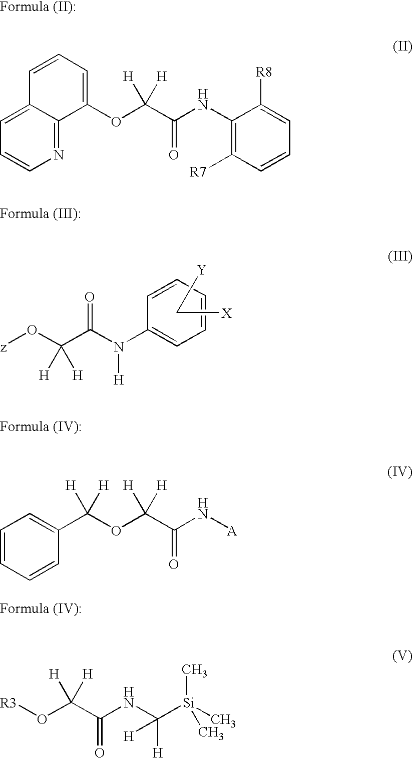
in which:
(i) R7 and R8 independently represent:
1) a hydrogen,
2) a halogen F or Cl,
3) one of the groups CF₃, CN, OR₄, SR₄, NHR₆, NR₆R′₆, OCOR₄, COR₄, CSR₄, COOR₄, SO₂R₄, SiR₄R′₄R″₄ and OCF₃, in which R₄, R′₄ and R″₄ independently are each a hydrogen or a C₁-C₂₀ alkyl radical optionally substituted with a phenyl radical and in which R₆ and R′₆ are each a C₁-C₂₀ alkyl radical optionally substituted with a phenyl radical,
4) a C₁-C₂₀ alkyl radical optionally substituted with a group OR₅ in which R₅ is a hydrogen or a C₁-C₂₀ alkyl radical,
5) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function;
(ii) Z is one of the following rings and heterocycles: 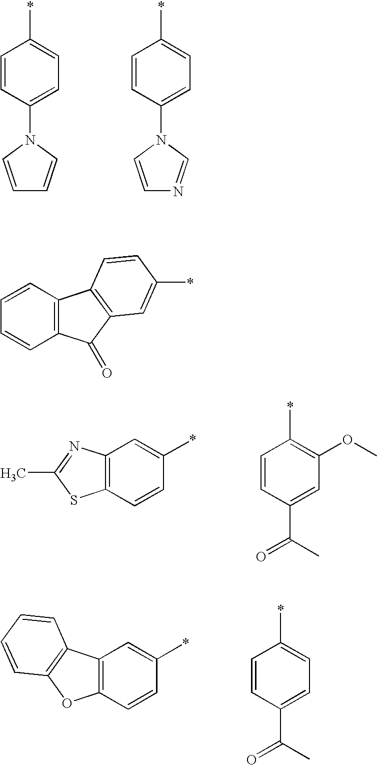
(iii) X and Y independently are each a hydrogen or A₂ or may form a fused ring C¹³ of 4 to 7 atoms, this ring optionally comprising at least one heteroatom C¹³, being optionally substituted with one or more substituents A₂, and optionally fused to another ring C¹⁴;
(iv) A is one of the following three groups: 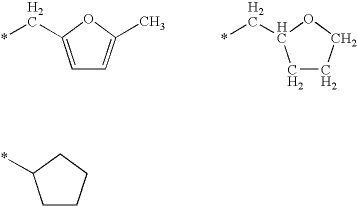
(v) R₃ has the same definition as for formula (I);
with the proviso that when Z is the dibenzofuran heterocycle, X and Y independently represent:
1) a hydrogen,
2) a halogen,
3) one of the groups CF₃, CN, OR₉, SR₄, NR₄R′₄, OCOR₄, COR₄, CSR₄, COOR₄, SO₂R₄, SiR₄R′₄R″₄, NO₂ and OCF₃, in which R₄, R′₄ and R″₄ independently are each a hydrogen or a C₁-C₂₀ alkyl radical optionally substituted with a phenyl radical and in which R₉ is a hydrogen or a C₂-C₂₀ alkyl radical optionally substituted with a phenyl radical,
4) a C₂-C₂₀ alkyl radical optionally substituted with a group OR₅,
5) a ring C¹¹ of 4 to 7 atoms optionally containing at least one heteroatom to form a heterocycle Hy₁₁ and/or being optionally fused to a ring C¹² of 4 to 7 atoms, these rings C¹¹ and C¹² optionally comprising a carbonyl or thiocarbonyl function; with the proviso that when Z is the ring: 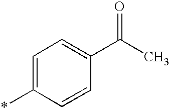
X and Y independently represent:
1) hydrogen, in which case X is different from Y,
2) fluorine,
3) one of the groups OR₁₀, SR₄, NR₄R′₄, OCOR₄, COR₄, CSR₄, COOR₆, SiR₄R′₄R″₄ and OCF₃, in which R₄, R′₄ and R″₄ independently are each a hydrogen or a C₁-C₂₀ alkyl radical optionally substituted with a phenyl radical, in which R₆ is a C₁-C₂₀ alkyl radical optionally substituted with a phenyl radical and in which R₁₀ is a hydrogen atom or a benzyl radical or a C₃-C₂₀ alkyl radical optionally substituted with a phenyl radical,
4) a C₂-C₂₀ alkyl radical optionally substituted with a group OR₅,
5) X and Y may also form a 5-atom ring C¹³ optionally containing one or two oxygen atoms and being optionally fused to another ring C¹⁴.

14. The regime or regimen as defined by claim 1, comprising administering at least one compound of formula (I) having one of the following formulae, or one of the corresponding salts and/or solvates thereof:

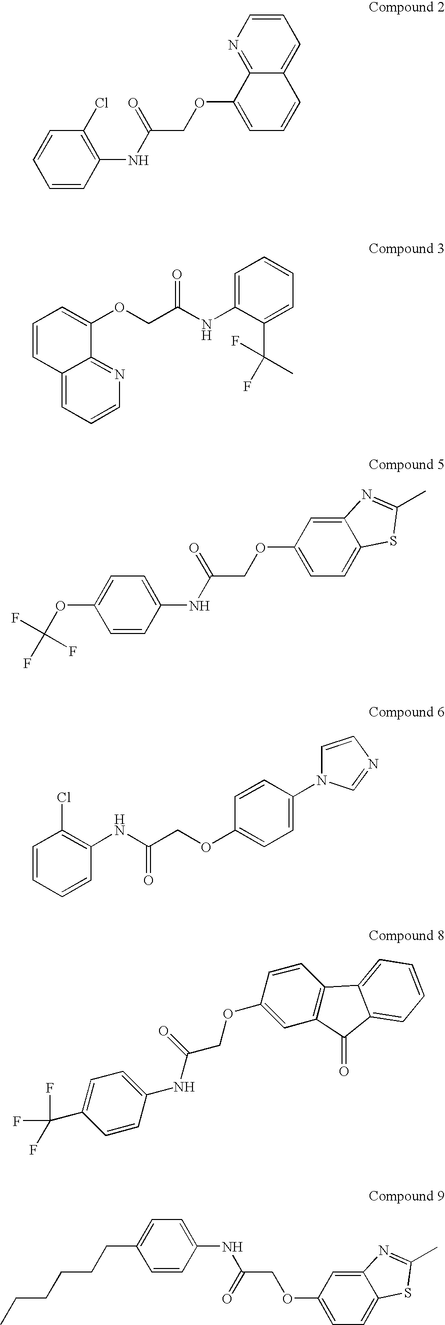

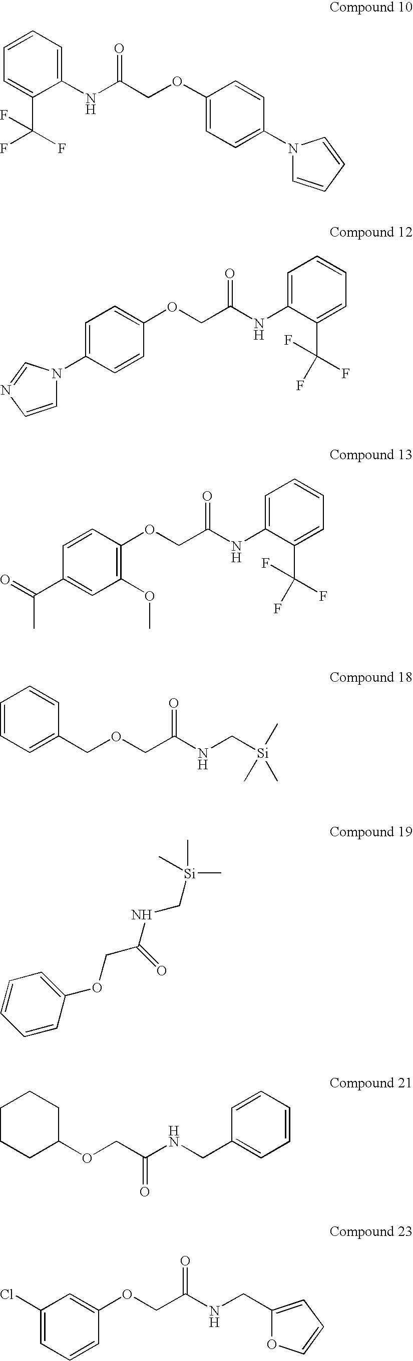

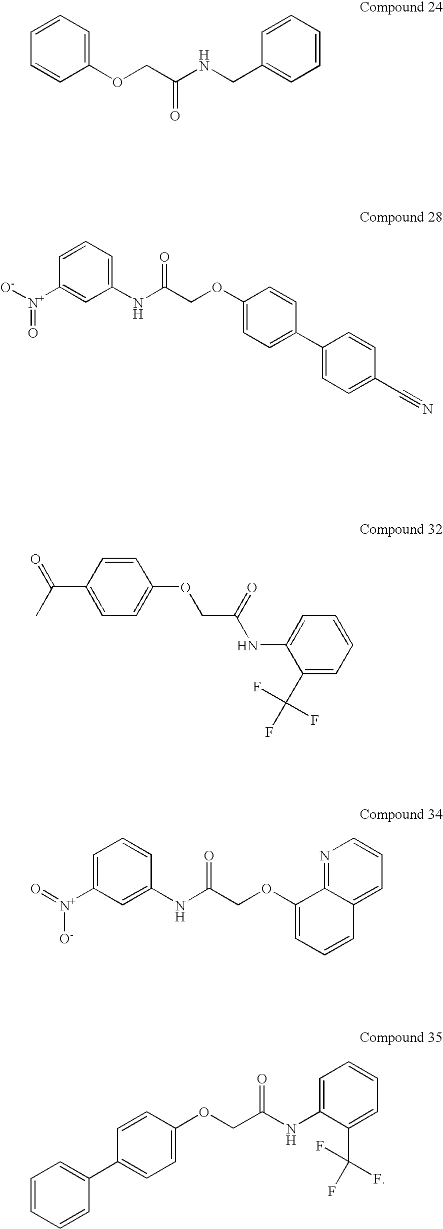

15. The regime or regimen as defined by claim 1, comprising administering to an individual a cosmetic composition which comprises from 10⁻³ to 10% by weight of said at least one compound of formula (I), or a salt and/or solvate thereof.

16. The regime or regimen as defined by claim 15, said composition being formulated for topical application.

17. The regime or regimen as defined by claim 15, said keratin materials comprising human hair, beard hair, moustache hair, eyelashes and/or eyebrows.

18. The regime or regimen as defined by claim 1, for preventing and/or limiting the canities of human hair, beard hair, moustache hair, eyelashes and/or eyebrows.

19. The regime or regimen as defined by claim 15, said cosmetic composition comprising a care and/or makeup composition for inducing and/or stimulating the pigmentation of keratin materials and/or for limiting their depigmentation and/or bleaching thereof.

20. The regime or regimen as defined by claim 19, said keratin materials comprising human hair, beard hair, moustache hair, eyelashes and/or eyebrows.

21. A cosmetic regime or regimen as defined by claim 1, for inducing and/or stimulating the pigmentation of keratin materials and/or for limiting the depigmentation and/or bleaching thereof, comprising topically applying onto the said keratin materials an effective amount of at least one 2-oxyacetamide compound of formula (I), or a salt and/or solvate thereof.

26. The cosmetic regime or regimen as defined by claim 21, said keratin materials comprising human hair, beard hair, moustache hair, eyelashes and/or eyebrows.

27. A cosmetic regime or regimen as defined by claim 1, for treating the canities of human hair, beard hair, moustache hair, the eyelashes and/or the eyebrows, comprising topically applying thereon an effective amount of said at least one 2-oxyacetamide compound of formula (I), or a salt and/or solvate thereof.
